SEQUENCE LISTING (13 sequences)

SEQ ID NO: 1 - protein, 190 aa, Homo sapiens
  MISC_FEATURE: Human Sclerostin
QGWQAFKNDATEIIPELGEYPEPPPELENNKTMNRAENGGRPPHHPFETKDVSEYSCRELHFTRYVTDGPCRSAKPVTELVCSGQCGPARLLPNAIGRGKWWRPSGPDFRCIPDRYRAQRVQLLCPGGEAPRARKVRLVASCKCKRLTRFHNQSELKDFGTEAARPQKGRKPRPRARSAKANQAELENAY

SEQ ID NO: 2 - protein, 5 aa, Mus musculus
  MISC_FEATURE: romo HCDR1
DYNMH

SEQ ID NO: 3 - protein, 17 aa, Mus musculus
  MISC_FEATURE: romo HCDR2
EINPNSGGAGYNQKFKG

SEQ ID NO: 4 - protein, 14 aa, Mus musculus
  MISC_FEATURE: romo HCDR3
LGYDDIYDDWYFDV

SEQ ID NO: 5 - protein, 11 aa, Mus musculus
  MISC_FEATURE: romoLCDR1
RASQDISNYLN

SEQ ID NO: 6 - protein, 7 aa, Mus musculus
  MISC_FEATURE: romo LCDR2
YTSRLLS

SEQ ID NO: 7 - protein, 9 aa, Mus musculus
  MISC_FEATURE: romo LCD3
QQGDTLPYT

SEQ ID NO: 8 - protein, 123 aa, Mus musculus [sequence omitted]

SEQ ID NO: 9 - protein, 107 aa, Mus musculus [sequence omitted]

SEQ ID NO: 10 - protein, 236 aa, Artificial Sequence
  Humanized antibody sequence
  MISC_FEATURE: romo light chain
MDMRVPAQLLGLLLLWLRGARCDIQMTQSPSSLSASVGDRVTITCRASQDISNYLNWYQQKPGKAPKLLIYYTSRLLSGVPSRFSGSGSGTDFTLTISSLQPEDFATYYCQQGDTLPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 468 aa, Artificial Sequence
  Humanized Antibody sequence
  MISC_FEATURE: romo heavy chain wild type
MDWTWRILFLVAAATGAHSEVQLVQSGAEVKKPGASVKVSCKASGYTFTDYNMHWVRQAPGQGLEWMGEINPNSGGAGYNQKFKGRVTMTTDTSTSTAYMELRSLRSDDTAVYYCARLGYDDIYDDWYFDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 12 - protein, 214 aa, Artificial Sequence
  Synthetic Polypeptide
  MISC_FEATURE: romo light chain wild type without signal sequence
DIQMTQSPSSLSASVGDRVTITCRASQDISNYLNWYQQKPGKAPKLLIYYTSRLLSGVPSRFSGSGSGTDFTLTISSLQPEDFATYYCQQGDTLPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 449 aa, Artificial Sequence
  Synthetic Polypeptide
  MISC_FEATURE: romo heavy chain wild type without signal sequence
EVQLVQSGAEVKKPGASVKVSCKASGYTFTDYNMHWVRQAPGQGLEWMGEINPNSGGAGYNQKFKGRVTMTTDTSTSTAYMELRSLRSDDTAVYYCARLGYDDIYDDWYFDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

(19) United States
(12) Patent Application Publication
(10) Pub. No.: US 2022/0275073 A1
(43) Pub. Date: Sep. 1, 2022

(54) Anti-Sclerostin Antibody Formulations

(71) Applicant: AMGEN INC., Thousand Oaks, CA (US)

(72) Inventor: Twinkle R. Christian, Thousand Oaks, CA (US)

(21) Appl. No.: 17/633,768

(22) PCT Filed: Aug. 7, 2020
(86) PCT No.: PCT/US20/45365

§ 371 Date: Feb. 8, 2022

Related U.S. Application Data
(60) Provisional application No. 62/885,672, filed on Aug. 12, 2019.

(51) Int. Cl. C07K 16/22; A61K 47/10; A61K 47/26; A61K 47/18

(52) U.S. Cl. CPC C07K 16/22; A61K 47/10; A61K 47/26; A61K 47/183

(57) ABSTRACT

The present disclosure is directed to pharmaceutical compositions comprising an anti-sclerostin antibody.

### CROSS-REFERENCE TO RELATED APPLICATIONS

The present application claims the benefit of priority to U.S. Provisional Application No. 62/885,672, filed on Aug. 12, 2019, the disclosure of which is incorporated by reference in its entirety.

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: ASCII (text) file named “53956_Seqlisting.txt”, 17,909 bytes, created on Aug. 7, 2020.

### INCORPORATION BY REFERENCE

The following applications are hereby incorporated by reference in their entirety: International Patent Application No. PCT/US2012/049331, filed Aug. 2, 2012, which claims priority to U.S. Provisional Patent Application No. 61/515,191, filed Aug. 4, 2011; U.S. patent application Ser. No. 11/410,540, filed Apr. 25, 2006, which claims priority to U.S. Provisional Patent Application No. 60/792,645, filed Apr. 17, 2006, U.S. Provisional Patent Application No. 60/782,244, filed Mar. 13, 2006, U.S. Provisional Patent Application No. 60/776,847, filed Feb. 24, 2006, and U.S. Provisional Patent Application No. 60/677,583, filed May 3, 2005; and U.S. patent application Ser. No. 11/411,003 (issued as U.S. Pat. No. 7,592,429), filed Apr. 25, 2006, which claims priority to U.S. Provisional Patent Application No. 60/792,645, filed Apr. 17, 2006, U.S. Provisional Patent Application No. 60/782,244, filed Mar. 13, 2006, U.S. Provisional Patent Application No. 60/776,847, filed Feb. 24, 2006, and U.S. Provisional Patent Application No. 60/677,583, filed May 3, 2005. The following applications also are hereby incorporated by reference: U.S. patent application Ser. No. 12/212,327, filed Sep. 17, 2008, which claims priority to U.S. Provisional Patent Application No. 60/973,024, filed Sep. 17, 2007; and U.S. patent application Ser. No. 12/811,171, filed Jun. 29, 2010, which is a U.S. National Phase Application pursuant to 35 U.S.C. § 371 of International Patent Application No. PCT/US08/86864, filed on Dec. 15, 2008, which claims priority to U.S. Provisional Patent Application No. 61/013,917, filed Dec. 14, 2007.

### BACKGROUND

### Field of the Invention

The present application is directed to pharmaceutical formulations comprising anti-sclerostin antibodies.

Protein-based pharmaceuticals are among the fastest growing therapeutic agents in (pre)clinical development and as commercial products. In comparison with small chemical drugs, protein pharmaceuticals have high specificity and activity at relatively low concentrations, and typically provide for therapy of high impact diseases such as various cancers, auto-immune diseases, and metabolic disorders (Roberts, Trends Biotechnol. 2014 Jul;32(7):372-80, Wang, Int J Pharm. 1999 Aug. 20;185(2):129-88).

Protein-based pharmaceuticals, such as recombinant proteins, can now be obtained in high purity when first manufactured due to advances in commercial scale purification processes. However, proteins are only marginally stable and are highly susceptible to degradation, both chemical and physical. Chemical degradation refers to modifications involving covalent bonds, such as deamidation, oxidation, cleavage or formation of new disulfide bridges, hydrolysis, isomerization, or deglycosylation. Physical degradation includes protein unfolding, undesirable adsorption to surfaces, and aggregation. Dealing with these physical and chemical instabilities is one of the most challenging tasks in the development of protein pharmaceuticals (Chi et al., Pharm Res, Vol. 20, No. 9, Sept 2003, pp. 1325-1336, Roberts, Trends Biotechnol. 2014 Jul.;32(7):372-80).

Protein aggregation represents a major event of physical instability of proteins and occurs due to the inherent tendency to minimize the thermodynamically unfavorable interaction between the solvent and hydrophobic protein residues. It can be particularly problematic because it is encountered during refolding, purification, sterilization, shipping, and storage processes. Aggregation can occur even under solution conditions where the protein native state is highly thermodynamically favored (e.g., neutral pH and 37° C.) and in the absence of stresses (Chi et al., Pharm Res, Vol. 20, No. 9, Sep. 2003, pp. 1325-1336, Roberts, Trends Biotechnol. 2014 Jul.;32(7):372-80, Wang, Int J Pharm. 1999 Aug 20;185(2):129-88, Mahler J Pharm Sci. 2009 Sep.;98(9):2909-34.).

Preserving protein stability and activity in biological and biotechnological applications poses serious challenges. There is a need in the art for optimized pharmaceutical compositions that provide for enhanced stabilization of therapeutic proteins and reduced aggregation and denaturation or degradation during formulation, filling, shipping, storage and administration, thereby preventing loss-of-function and adverse immunogenic reactions.

### SUMMARY

In one aspect, described herein is a pharmaceutical composition comprising an anti-sclerostin antibody; a buffer comprising glutamic acid, histidine or succinic acid; and a polyol, wherein the pharmaceutical composition comprises a pH of pH4-pH7.

In some embodiments, the buffer is present at a concentration of about 10 mM to about 50 mM. In some embodiments, the polyol is present in an amount of about 1% to about 10% w/v. In some embodiments, the polyol is sorbitol and is present in an amount of about 5% to about 10% w/v. In some embodiments, the sorbitol is present in an amount of about 5% w/v.

In some embodiments, the pharmaceutical composition further comprises glycerol (e.g., in an amount of about 1% to about 5% w/v).

In some embodiments, the pharmaceutical composition further comprises sucrose (e.g., in an amount of about 1% to about 10% w/v).

In some embodiments, the pharmaceutical composition further comprises an amino acid other than histidine. In some embodiments, the amino acid is arginine. In some embodiments, the arginine is present in an amount ranging from 10 mM to about 250 mM. In some embodiments, the amino acid is methionine. In some embodiments, the methionine is present in an amount of about 10 mM to about 100 mM.

In some embodiments, the pharmaceutical composition further comprises a surfactant. In some embodiments, the surfactant is polysorbate 20, polysorbate 80, F16 or Triton.

In some embodiments, the pharmaceutical composition comprises an anti-sclerostin antibody at a concentration of at least 70 mg/mL. In some embodiments, the pharmaceutical composition comprises an anti-sclerostin antibody at a concentration of about 70 mg/mL to about 210 mg/mL.

In some embodiments, the anti-sclerostin antibody is romosozumab.

In some embodiments, the pharmaceutical composition comprises 10 mM glutamic acid and 5% sorbitol at pH 4.5. In some embodiments, the pharmaceutical composition comprises 10 mM glutamic acid and 5% sorbitol at pH 5.2. In some embodiments, the pharmaceutical composition comprises 10 mM succinic acid and 5% sorbitol at pH 5.2. In some embodiments, the pharmaceutical composition comprises 10 mM histidine and 5% sorbitol at pH 6.

It should be understood that while various embodiments in the specification are presented using “comprising” language, under various circumstances, a related embodiment may also be described using “consisting of” or “consisting essentially of” language. It is to be noted that the term “a” or “an”, refers to one or more, for example, “an immunoglobulin molecule,” is understood to represent one or more immunoglobulin molecules. As such, the terms “a” (or “an”), “one or more,” and “at least one” can be used interchangeably herein.

It should also be understood that when describing a range of values, the characteristic being described could be an individual value found within the range. For example, “a pH from about pH 4 to about pH 6,” could be, but is not limited to, pH 4, 4.2, 4.6, 5.1, 5.5, etc. and any value in between such values. Additionally, “a pH from about pH 4 to about pH 6,” should not be construed to mean that the pH of a formulation in question varies 2 pH units in the range from pH 4 to pH 6 during storage, but rather a value may be picked in that range for the pH of the solution, and the pH remains buffered at about that pH. In some embodiments, when the term “about” is used, it means the recited number plus or minus 5%, 10%, 15% or more of that recited number. The actual variation intended is determinable from the context.

In any of the ranges described herein, the endpoints of the range are included in the range. However, the description also contemplates the same ranges in which the lower and/or the higher endpoint is excluded. Additional features and variations of the invention will be apparent to those skilled in the art from the entirety of this application, including the drawing and detailed description, and all such features are intended as aspects of the invention. Likewise, features of the description described herein can be re-combined into additional embodiments that also are intended as aspects of the invention, irrespective of whether the combination of features is specifically mentioned above as an aspect or embodiment of the invention. Also, only such limitations which are described herein as critical to the invention should be viewed as such; variations of the invention lacking limitations which have not been described herein as critical are intended as aspects of the invention.

### BRIEF DESCRIPTION OF THE FIGURES

FIG. 1 is a graph showing romosozumab high molecular weight (HMW) peak area percents in various formulations that were stored at 4° C. for up to 24 months.

FIG. 2 is a graph showing romosozumab high molecular weight (HMW) peak area percents in various formulations that were stored at 37° C. for up to 4 weeks.

FIG. 3 is a graph showing romosozumab high molecular weight (HMW) peak area percents in various formulations that were stored at 45° C. for up to 4 weeks.

FIG. 4 is a graph showing main peak area (%) of romosozumab in various formulations when stored at 4° C. for up to 24 months as assessed by cation-exchange HPLC.

FIG. 5 is a graph showing main peak area (%) of romosozumab in various formulations when stored at −70° C. for up to 24 months as assessed by cation-exchange HPLC.

FIG. 6 is a graph showing main peak area (%) of romosozumab in various formulations when stored at 4° C., −30° C. and −70° C. for up to 24 months as assessed by cation-exchange HPLC.

FIG. 7 is a graph showing main peak area (%) of romosozumab in various formulations when stored at 4° C., 25° C., 37° C., 45° C., −30° C. and −70° C. for up to 4 weeks as assessed by cation-exchange HPLC.

FIG. 8 is a graph showing acid peak area (%) of romosozumab in various formulations when stored at 4° C., 25° C., 37° C., 45° C., −30° C. and −70° C. for up to 4 weeks as assessed by cation-exchange HPLC.

FIG. 9 is a graph showing acid peak area (%) of romosozumab in various formulations when stored at 4° C., −30° C. and −70° C. for up to 24 months as assessed by cation-exchange HPLC.

FIG. 10 is a graph showing acid peak area (%) of romosozumab in various formulations when stored at 4° C., −30° C. and −70° C. for up to 24 months as assessed by cation-exchange HPLC.

FIG. 11 is a chromatogram of romosozumab in formulation Formulation 4 after 3 months storage at 4° C., 25° C., and 37° C. as assessed by cation exchange-HPLC.

FIG. 12 is a graph showing the main peak area (%) of romosozumab in various formulations when stored at 4° C., −30° C. and −70° C. for two years.

FIG. 13 is a graph showing the acidic peak area (%) of romosozumab in various formulations when stored at 4° C., −30° C. and −70° C. for two years as assessed by cation exchange HPLC.

FIG. 14 is a graph showing the basic peak stability of romosozumab in various formulations when stored at 4° C., −30° C. and −70° C. for two years as assessed by cation exchange HPLC.

FIG. 15 is a graph showing percent high molecular weight species of romosozumab in various formulations when stored at 4° C. for various time points (4 weeks, 3 months, 6 months, 1 year, 1.5 years, and 2 years) as assessed by capillary electrophoresis-SDS

FIG. 16 is a graph showing the results of the high concentration syringe study (70 mg/mL romosozumab in various formulations) at time 0 as assessed by HIAC.

FIG. 17 is a graph showing the results of the high concentration syringe study (70 mg/mL romosozumab in various formulations) at the 2 year time point as assessed by HIAC.

FIG. 18 is a graph showing the results of the high concentration syringe study (120 mg/mL romosozumab in various formulations) at time 0 as assessed by HIAC.

FIG. 19 is a graph showing the results of the high concentration syringe study (120 mg/mL romosozumab in various formulations) at the 2 year time point as assessed by HIAC.

### DETAILED DESCRIPTION

The present disclosure describes formulations comprising an anti-sclerostin antibody. Various aspects of the formulation are described below. The use of section headings are merely for the convenience of reading, and not intended to be limiting per se. The entire document is intended to be viewed as a unified disclosure, and it should be understood that all combinations of features described herein are contemplated.

In one aspect, described herein is a pharmaceutical formulation comprising (a) an anti-sclerostin antibody; (b) a buffer comprising glutamic acid, histidine or succinic acid; and (c) a polyol; wherein the pharmaceutical composition comprises a pH of pH4-pH7. As demonstrated in the Examples, formulations comprising the combination of components described herein are stable under a variety of conditions for extended period of time (up to two years) at a range of temperatures (e.g., −30° C., −70° C. and 4° C.).

### Stability

The terms “stability” and “stable” as used herein in the context of a composition comprising an antibody (or antigen binding fragment thereof) refer to the resistance of the antibody (or antigen binding fragment thereof) in the composition to aggregation, degradation or fragmentation under given manufacture, preparation, transportation and/or storage conditions. Antibody formulations comprising a high degree of stability demonstrate enhanced reliability and safety and, as such, are advantageous for clinical use.

Antibody stability in a composition is optionally assessed by examining a desired parameter of the antibody in the composition (e.g., aggregation, degradation of heavy and/or light chains, chemical modification, etc.) over time. In this regard, a parameter is typically examined at an initial time point (T0) and an assessment time point (T1), optionally while exposing the antibody to any of a number of environmental conditions, and compared. An initial time point can be, for instance, the time that the antibody is first formulated in a composition or first examined for quality (i.e., examined to determine whether the antibody composition meets regulatory or manufacturing specifications with respect to aggregation or degradation). An initial time point also can be the time at which the antibody is reformulated in a composition (e.g., reformulated at a higher or lower concentration compared to an initial preparation). An assessment time point is, in various embodiments, about 1 week (or about 2 weeks, or about 3 weeks, or about 4 weeks, or about 5 weeks, or about 6 weeks, or about 7 weeks, or about 8 weeks, or about 10 weeks, or about 3 months, or about 6 months or about 1 year) after the initial time point. The desired parameter (e.g., aggregation or degradation) of the antibody or fragment thereof in the composition can be assessed under a variety of storage conditions, such as temperatures of −30° C., 4° C., 20° C. or 40° C., shaking, pH, storage in different container materials (e.g., glass vials, pre-filled syringes, etc.), and the like.

Exemplary methods for determining the degree of aggregation and/or types and/or sizes of aggregates present in a composition comprising the antibody include, but are not limited to, size exclusion chromatography (SEC), high performance size exclusion chromatography (HPSEC), static light scattering (SLS), Fourier Transform Infrared Spectroscopy (FTIR), circular dichroism (CD), urea-induced protein unfolding techniques, intrinsic tryptophan fluorescence, differential scanning calorimetry, and 1-anilino-8-naphthalenesulfonic acid (ANS) protein binding techniques. Size exclusion chromatography (SEC) may be performed to separate molecules on the basis of their size, by passing the molecules over a column packed with the appropriate resin, the larger molecules (e.g. aggregates) will elute before smaller molecules (e.g., monomers). The molecules are generally detected by UV absorbance at 280 nm and may be collected for further characterization. High pressure liquid chromatographic columns are often utilized for SEC analysis (HP-SEC). Alternatively, analytical ultracentrifugation (AUC) may be utilized. AUC is an orthogonal technique which determines the sedimentation coefficients of macromolecules in a liquid sample. Like SEC, AUC is capable of separating and detecting antibody fragments/aggregates from monomers and is further able to provide information on molecular mass. Antibody aggregation in a composition may also be characterized by particle counter analysis using a coulter counter or by turbidity measurements using a turbidimeter. Turbidity is a measure of the amount by which the particles in a solution scatter light and, thus, may be used as a general indicator of protein aggregation. In addition, non-reducing polyacrylamide gel electrophoresis (PAGE) or capillary gel electrophoresis (CGE) may be used to characterize the aggregation and/or fragmentation state of antibodies or antibody fragments in a composition.

Exemplary methods for determining antibody degradation include, but are not limited to, size-exclusion chromatography (SEC), sodium dodecyl sulfate-polyacrylamide gel electrophoresis (SDS-PAGE) and capillary electrophoresis with SDS (CE-SDS) and reversed phase HPLC with in-line MS detection.

In various embodiments, less than 5% of the antibody described herein in the composition is in aggregate form under conditions of interest. For instance, less than 4%, or less than 3%, or less than 2%, or less than 1% of the antibody in the composition is in aggregate form after storage at −30° C., 4° C., 20° C. or 40° C. for a period of about 1 week (or about 2 weeks, or about 3 weeks, or about 4 weeks, or about 5 weeks, or about 6 weeks, or about 7 weeks, or about 8 weeks, or about 10 weeks, or about 3 months, or about 6 months or about 1 year). In some embodiments, less than 5% (or less than 4% or less than 3% or less than 2% or less than 1% or less) of the antibody described herein in the composition is in aggregate form after storage for two weeks at about 4° C.

For example at least 85% (or at least 90%, or at least 91%, or at least 92%, or at least 93%, or at least 94%, or at least 95%, or at least 96%, or at least 97%, or at least 98%, or at least 99%) of antibody in a composition optionally is present in non-aggregate (i.e., monomeric) form after storage at −30° C., 4° C., 20° C. or 40° C. for a period of about 1 week (or about 2 weeks, or about 3 weeks, or about 4 weeks, or about 5 weeks, or about 6 weeks, or about 7 weeks, or about 8 weeks, or about 10 weeks, or about 3 months, or about 6 months or about 1 year). In some embodiments, at least 85% (or at least 90%, or at least 91%, or at least 92%, or at least 93%, or at least 94%, or at least 95%, or at least 96%, or at least 97%, or at least 98%, or at least 99% or more) of the antibody is present in the composition in non-aggregate form after two weeks of storage at about 4° C. In some embodiments, at least 99% of the antibody is present in the composition in non-aggregate form after storage for two weeks at about 4° C. for two weeks and/or at least 95% of antibody present in the composition is in non-aggregate form after storage for two weeks at 40° C.

In various embodiments, less than 5% of the antibody described herein in the composition is degraded. For instance, less than 4%, or less than 3%, or less than 2%, or less than 1% or less of the antibody in the composition is degraded under conditions of interest. For example, optionally at least 85% (or at least 90%, or at least 91%, or at least 92%, or at least 93%, or at least 94%, or at least 95%, or at least 96%, or at least 97%, or at least 98%, or at least 99%) of the antibody is intact (i.e., not degraded) in a composition stored at about −30° C., about 4° C., about 20° C. or about 40° C. for a period of about 1 week (or about 2 weeks, or about 3 weeks, or about 4 weeks, or about 5 weeks, or about 6 weeks, or about 7 weeks, or about 8 weeks, or about 10 weeks, or about 3 months, or about 6 months or about 1 year). In some aspects, at least 85% (or at least 90%, or at least 91%, or at least 92%, or at least 93%, or at least 94%, or at least 95%, or at least 96%, or at least 97%, or at least 98%, or at least 99% or more) of the antibody is intact (i.e., non-degraded) after storage in a composition at about 4° C. for a period of two weeks. In some embodiments, at least 99% of the antibody remains intact when stored in a composition at about 4° C. for two weeks and/or at least 95% remains intact when stored in a composition at about 40° C. for two weeks.

Functional or activity stability of the antibody in a composition also is contemplated herein. Assays for detecting and/or quantifying, e.g., antibody binding to a target or sclerostin neutralization are known in the art. Optionally, the antibody demonstrates about 50-100% activity under conditions of interest compared to the activity of the antibody at the initial time point. For example, the antibody retains a level of activity of between about 60-90% or 70-80% compared to the activity the initial time point. Accordingly, functional stability of the antibody includes retention of activity of at least about 50%, 55%, 60%, 65%, 70%, 75%, 80%, 85%, 90%, 95% or 100% and can include activity measurements greater than 100% such as 105%, 110%, 115%, 120%, 125% or 150% or more compared to the activity at the initial time point.

### Buffers

The pharmaceutical formulation described herein comprises a buffer, which optionally may be selected from the group consisting of histidine, glutamic acid and succinic acid, and combinations thereof. In some embodiments, the pharmaceutical composition comprises at least one buffer selected from the group consisting of histidine, glutamic acid and succinic acid and combinations thereof.

Buffering agents are often employed to control pH in the formulation. In some embodiments, the buffer is added in a concentration that maintains pH of the formulation of about 4 to 7, or about 4.5 to 6, or about 5.2. The effect of pH on formulations may be characterized using any one or more of several approaches such as accelerated stability studies and calorimetric screening studies (Remmele R. L. Jr., et al., Biochemistry, 38(16): 5241-7 (1999)).

Organic acids, phosphates and Tris are suitable buffers in protein formulations (Table 1). The buffer capacity of the buffering species is maximal at a pH equal to the pKa and decreases as pH increases or decreases away from this value. Ninety percent of the buffering capacity exists within one pH unit of its pKa. Buffer capacity also increases proportionally with increasing buffer concentration.

Several factors are typically considered when choosing a buffer. For example, the buffer species and its concentration should be defined based on its pKa and the desired formulation pH. Also, the buffer is preferably compatible with the protein drug, other formulation excipients, and does not catalyze any degradation reactions. Polyanionic carboxylate buffers such as citrate and succinate may be able to form covalent adducts with the side chain residues of proteins. A third aspect to be considered is the sensation of stinging and irritation the buffer may induce. For example, citrate is known to cause stinging upon injection (Laursen T, et al., Basic Clin Pharmacol Toxicol., 98(2): 218-21 (2006)). The potential for stinging and irritation is greater for drugs that are administered via the SC or IM routes, where the drug solution remains at the site for a relatively longer period of time than when administered by the IV route where the formulation gets diluted rapidly into the blood upon administration. For formulations that are administered by direct IV infusion, the total amount of buffer (and any other formulation component) needs to be monitored. For example, it has been reported that potassium ions administered in the form of the potassium phosphate buffer, can induce cardiovascular effects in a patient (Hollander-Rodriguez J C, et al., Am. Fam. Physician., 73(2): 283-90 (2006)).

TABLE 1
Buffering agents and their pK values

| Buffer | pKₐ |
|---|---|
| Acetate | 4.8 |
| Succinate | pKₐ₁ = 4.8, pKₐ₂ = 5.5 |
| Citrate | pKₐ₁ = 3.1, pKₐ₂ = 4.8, pKₐ₃ = 6.4 |
| Histidine | 6.0 |
| (imidazole) | |
| Phosphate | pKₐ₁ = 2.15, pKₐ₂ = 7.2, pKₐ₃ = 12.3 |
| Tris | 8.1 |

The buffer system present in the formulation is selected to be physiologically compatible and to maintain a desired pH.

The buffer may be present in any amount suitable to maintain the pH of the formulation at a predetermined level. The buffer may be present at a concentration between about 0.1 mM and about 1000 mM (1 M), or between about 5 mM and about 200 mM, or between about 5 mM to about 100 mM, or between about 10 mM and 50 about mM. Suitable buffer concentrations encompass concentrations of about 200 mM or less. In some embodiments, the buffer in the formulation is present in a concentration of about 190 mM, about 180 mM, about 170 mM, about 160 mM, about 150 mM, about 140 mM, about 130 mM, about 120 mM, about 110 mM, about 100 mM, about 80 mM, about 70 mM, about 60 mM, about 50 mM, about 40 mM, about 30 mM, about 20 mM, about 10 mM or about 5 mM.

In some embodiments, the concentration of the buffer is at least 0.1, 0.5, 0.7, 0.8, 0.9, 1.0, 1.2, 1.5, 1.7, 2, 3, 4, 5, 6, 7, 8, 9, 10, 11, 12, 13, 14, 15, 16, 17, 18, 19, 20, 30, 40, 50, 60, 70, 80, 90, 100, 200, 500, 700, or 900 mM. In some embodiments, the concentration of the buffer is between 1, 1.2, 1.5, 1.7, 2, 3, 4, 5, 6, 7, 8, 9, 10, 11, 12, 13, 14, 15, 16, 17, 18, 19, 20, 30, 40, 50, 60, 70, 80, or 90 mM and 100 mM. In some embodiments, the concentration of the buffer is between 5, 6, 7, 8, 9, 10, 11, 12, 13, 14, 15, 16, 17, 18, 19, 20, 30, or 40 mM and 50 mM. In some embodiments, the concentration of the buffer is about 10 mM.

### Surfactants

The pharmaceutical compositions described here comprise at least one surfactant. Surfactants are commonly used in protein formulations to prevent surface-induced degradation. Surfactants are amphipathic molecules with the capability of out-competing proteins for interfacial positions. Hydrophobic portions of the surfactant molecules occupy interfacial positions (e.g., air/liquid), while hydrophilic portions of the molecules remain oriented towards the bulk solvent. At sufficient concentrations (typically around the detergent's critical micellar concentration), a surface layer of surfactant molecules serve to prevent protein molecules from adsorbing at the interface. Thereby, surface-induced degradation is minimized. Surfactants include, e.g., fatty acid esters of sorbitan polyethoxylates, i.e., polysorbate 20 and polysorbate 80 (see e.g., Avonex®, Neupogen®, Neulasta®). The two differ only in the length of the aliphatic chain that imparts hydrophobic character to the molecules, C-12 and C-18, respectively. Accordingly, polysorbate-80 is more surface-active and has a lower critical micellar concentration than polysorbate-20. The surfactant poloxamer 188 has also been used in several marketed liquid products such Gonal-F®, Norditropin®, and Ovidrel®.

Detergents can also affect the thermodynamic conformational stability of proteins. Here again, the effects of a given excipient may be protein specific. For example, polysorbates may reduce the stability of some proteins and increase the stability of others. Detergent destabilization of proteins can be rationalized in terms of the hydrophobic tails of the detergent molecules that can engage in specific binding with partially or wholly unfolded protein states. These types of interactions could cause a shift in the conformational equilibrium towards the more expanded protein states (i.e., increasing the exposure of hydrophobic portions of the protein molecule in complement to binding polysorbate). Alternatively, if the protein native state exhibits some hydrophobic surfaces, detergent binding to the native state may stabilize that conformation.

Another aspect of polysorbates is that they are inherently susceptible to oxidative degradation. Often, as raw materials, they contain sufficient quantities of peroxides to cause oxidation of protein residue side-chains, especially methionine. The potential for oxidative damage arising from the addition of stabilizer emphasizes the point that the lowest effective concentrations of excipients should be used in formulations. For surfactants, the effective concentration for a given protein will depend on the mechanism of stabilization. It has been postulated that if the mechanism of surfactant stabilization is related to preventing surface-denaturation the effective concentration will be around the detergent's critical micellar concentration. Conversely, if the mechanism of stabilization is associated with specific protein-detergent interactions, the effective surfactant concentration will be related to the protein concentration and the stoichiometry of the interaction (Randolph T. W., et al., Pharm Biotechnol., 13:159-75 (2002)).

Surfactants may also be added in appropriate amounts to prevent surface related aggregation phenomenon during freezing and drying (Chang, B, J. Pharm. Sci. 85:1325, (1996)). Exemplary surfactants include anionic, cationic, nonionic, zwitterionic, and amphoteric surfactants including surfactants derived from naturally-occurring amino acids. Anionic surfactants include, but are not limited to, sodium lauryl sulfate, dioctyl sodium sulfosuccinate and dioctyl sodium sulfonate, chenodeoxycholic acid, N-lauroylsarcosine sodium salt, lithium dodecyl sulfate, 1-octanesulfonic acid sodium salt, sodium cholate hydrate, sodium deoxycholate, and glycodeoxycholic acid sodium salt. Cationic surfactants include, but are not limited to, benzalkonium chloride or benzethonium chloride, cetylpyridinium chloride monohydrate, and hexadecyltrimethylammonium bromide. Zwitterionic surfactants include, but are not limited to, CHAPS, CHAPSO, SB3-10, and SB3-12. Non-ionic surfactants include, but are not limited to, digitonin, Triton X-100, Triton X-114, TWEEN-20, and TWEEN-80. In another embodiment, surfactants include lauromacrogol 400; polyoxyl 40 stearate; polyoxyethylene hydrogenated castor oil 10, 40, 50 and 60; glycerol monostearate; polysorbate 40, 60, 65 and 80; soy lecithin and other phospholipids such as DOPC, DMPG, DMPC, and DOPG; sucrose fatty acid ester; methyl cellulose and carboxymethyl cellulose.

Pharmaceutical compositions described herein comprise at least one surfactant, either individually or as a mixture in different ratios. In some embodiments, the composition comprises a surfactant at a concentration of about 0.001% to about 5% w/v (or about 0.004 to about 0.5% w/v or about 0.001 to about 0.01% w/v or about 0.004 to about 0.01% w/v). In some embodiments, the composition comprises a surfactant at a concentration of at least 0.001, at least 0.002, at least 0.003, at least 0.004, at least 0.005, at least 0.007, at least 0.01, at least 0.05, at least 0.1, at least 0.2, at least 0.3, at least 0.4, at least 0.5, at least 0.6, at least 0.7, at least 0.8, at least 0.9, at least 1.0, at least 1.5, at least 2.0, at least 2.5, at least 3.0, at least 3.5, at least 4.0, or at least 4.5% w/v. In some embodiments, the composition comprises a surfactant at a concentration of about 0.004% to about 0.5% w/v. In some embodiments, the composition comprises a surfactant at a concentration of about 0.004 to about 0.5% w/v. In some embodiments, the composition comprises a surfactant at a concentration of about 0.001 to about 0.01% w/v. In some embodiments, the composition comprises a surfactant at a concentration of about 0.004 to about 0.01% w/v. In some embodiments, the composition comprises a surfactant at a concentration of about 0.004, about 0.005, about 0.007, about 0.01, about 0.05, about 0.1, about 0.2, about 0.3, about 0.4% w/v to about 0.5% w/v. In some embodiments, the composition comprises a surfactant incorporated in a concentration of about 0.001% to about 0.01% w/v.

### Saccharides

The pharmaceutical compositions described herein comprise at least one saccharide. A saccharide can be added as a stabilizer or a bulking agent. The term “stabilizer” as used herein refers to an excipient capable of preventing aggregation or other physical degradation, as well as chemical degradation (for example, autolysis, deamidation, oxidation, etc.) in an aqueous and solid state. Stabilizers that are employed in pharmaceutical compositions include, but are not limited to, sucrose, trehalose, mannose, maltose, lactose, glucose, raffinose, cellobiose, gentiobiose, isomaltose, arabinose, glucosamine, fructose, mannitol, sorbitol, glycine, arginine HCL, poly-hydroxy compounds, including polysaccharides such as dextran, starch, hydroxyethyl starch, cyclodextrins, N-methyl pyrollidene, cellulose and hyaluronic acid, and sodium chloride (Carpenter et al., Develop. Biol. Standard 74:225, (1991)).

In some embodiments, the at least one saccharide is selected from the group consisting of monosaccharide, disaccharide, cyclic polysaccharide, sugar alcohol, linear branched dextran, and linear non-branched dextran, or a combination thereof. In some embodiments, the at least one saccharide is a disaccharide selected from the group consisting of sucrose, trehalose, mannitol, and sorbitol or a combination thereof.

In some embodiments, the pharmaceutical composition comprises at least one saccharide at a concentration of about 0.01% to about 40% w/v, or about 0.1% to about 20% w/v, or about 1% to about 15% w/v. In some embodiments, the pharmaceutical composition comprises at least one saccharide at a concentration of at least 0.5, at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 30, or at least 40% w/v. In some embodiments, the pharmaceutical composition comprises at least one saccharide at a concentration of about 1%, about 2%, about 3%, about 4%, about 5%, about 6%, about 7%, about 8%, about 9%, about 10%, about 11%, about 12%, about 13%, about 14% to about 15% w/v. In some embodiments, the pharmaceutical composition comprises at least one saccharide at a concentration of about 1% to about 15% w/v. In a yet further embodiment, the pharmaceutical composition comprises at least one saccharide at a concentration of about 9%, about 9.5%, about 10%, about 10.5%, about 11%, about 11.5%, or about 12% w/v. In some embodiments, the pharmaceutical composition comprises at least one saccharide at a concentration of about 9% to about 12% w/v. In some embodiments, the at least one saccharide is in the composition at a concentration of about 9% w/v. In some embodiments, the at least one saccharide is sorbitol, sucrose, trehalose or mannitol or a combination thereof.

In some embodiments, the formulation comprises sorbitol in an amount of about 1%, about 2%, about 3%, about 4%, about 5%, about 6%, about 7%, about 8%, about 9%, about 10%, about 11%, or about 12%. In some embodiments, the formulation comprises sorbitol in an amount of about 5%, about 6%, about 7%, about 8%, about 9%, or about 10%. In some embodiments, the formulation comprises sorbitol in an amount of about 5%.

In some embodiments, the formulation further comprises sucrose and is present in the composition ranging from 1%, about 2%, about 3%, about 4%, about 5%, about 6%, about 7%, about 8%, about 9%, about 10%, about 11%, about 12%, about 13%, about 14% to about 15% w/v. In some embodiments, the formulation further comprises sucrose in an amount of about 9%.

In some embodiments, the formulation further comprises glycerol. In some embodiments, the formulation further comprises glycerol in an amount of about 1%, about 2%, about 3%, about 4%, or about 5%. The formulation optionally further comprises glycerol in an amount of about 1% or about 2.5%.

If desired, the formulations also include appropriate amounts of bulking and osmolarity regulating agents, such as a saccharide, suitable for forming a lyophilized “cake.”

In some embodiments, the formulation further comprises glycerol. In some embodiments. The formulation further comprises glycerol in an amount of about 1%, about 2%, about 3%, about 4%, or about 5%. The formulation further comprises glycerol in an amount of about 1% or about 2.5%.

In some embodiments, the formulation comprises 10 mM glutamic acid and 5% sorbitol at pH 4.5.

In some embodiments, the formulation comprises 10 mM glutamic acid and 5% sorbitol at pH 5.2.

In some embodiments, the formulation comprises 10 mM succinic acid and 5% sorbitol at pH 5.2.

In some embodiments, the formulation comprises 10 mM histidine and 5% sorbitol at pH 6.

### Other Considerations

As used herein, the term “pharmaceutical composition” relates to a composition which is suitable for administration to a subject in need thereof. The terms “subject” or “individual” or “animal” or “patient” are used interchangeably herein to refer to any subject, particularly a mammalian subject, for whom administration of the pharmaceutical composition of the invention is desired. Mammalian subjects include humans, non-human primates, dogs, cats, guinea pigs, rabbits, rats, mice, horses, cattle, cows, and the like, with humans being preferred. The pharmaceutical composition of the present disclosure is stable and pharmaceutically acceptable, i.e., capable of eliciting the desired therapeutic effect without causing significant undesirable local or systemic effects in the subject to which the pharmaceutical composition is administered. Pharmaceutically acceptable compositions of the disclosure may be sterile and/or pharmaceutically inert. Specifically, the term “pharmaceutically acceptable” can mean approved by a regulatory agency or other generally recognized pharmacopoeia for use in animals, and more particularly in humans.

The formulation provided by the disclosure comprises an antibody described herein. In some embodiments, the antibody is provided in a therapeutically effective amount. By “therapeutically effective amount” is meant an amount of said heterodimeric antibody that elicits the desired therapeutic effect. Therapeutic efficacy and toxicity can be determined by standard pharmaceutical procedures in cell cultures or experimental animals, e.g., ED50 (the dose therapeutically effective in 50% of the population) and LD50 (the dose lethal to 50% of the population). The dose ratio between therapeutic and toxic effects is the therapeutic index, and it can be expressed as the ratio, ED50/LD50. Formulations that exhibit large therapeutic indices are generally preferred.

Protein formulations are generally administered parenterally. When given parenterally, they must be sterile. Sterile diluents include liquids that are pharmaceutically acceptable (safe and non-toxic for administration to a human) and useful for the preparation of a liquid formulation, such as a formulation reconstituted after lyophilization. Exemplary diluents include sterile water, bacteriostatic water for injection (BWFI), a pH buffered solution (e.g., phosphate-buffered saline), sterile saline solution, Ringer's solution or dextrose solution. Diluents can include aqueous solutions of salts and/or buffers.

Excipients are additives that are included in a formulation because they either impart or enhance the stability, delivery and manufacturability of a drug product. Regardless of the reason for their inclusion, excipients are an integral component of a drug product and therefore need to be safe and well tolerated by patients. For protein drugs, the choice of excipients is particularly important because they can affect both efficacy and immunogenicity of the drug. Hence, protein formulations need to be developed with appropriate selection of excipients that afford suitable stability, safety, and marketability.

The excipients described herein are organized either by their chemical type or their functional role in formulations. Brief descriptions of the modes of stabilization are provided when discussing each excipient type. Given the teachings and guidance provided herein, those skilled in the art will readily be able to vary the amount or range of excipient without increasing viscosity to an undesirable level. Excipients may be chosen to achieve a desired osmolality (i.e., isotonic, hypotonic or hypertonic) of the final solution, pH, desired stability, resistance to aggregation or degradation or precipitation, protection under conditions of freezing, lyophilization or high temperatures, or other properties. A variety of types of excipients are known in the art. Exemplary excipients include salts, amino acids, other tonicity agents, surfactants, stabilizers, bulking agents, cryoprotectants, lyoprotectants, anti-oxidants, metal ions, chelating agents and/or preservatives.

Further, where a particular excipient is reported in a formulation by, e.g., percent (%) w/v, those skilled in the art will recognize that the equivalent molar concentration of that excipient is also contemplated.

### Other Stabilizers and Bulking Agents

Stabilizers include a class of compounds that can serve as cryoprotectants, lyoprotectants, and glass forming agents. Cryoprotectants act to stabilize proteins during freezing or in the frozen state at low temperatures. Lyoprotectants stabilize proteins in the freeze-dried solid dosage form by preserving the native-like conformational properties of the protein during dehydration stages of freeze-drying. Glassy state properties have been classified as “strong” or “fragile” depending on their relaxation properties as a function of temperature. It is important that cryoprotectants, lyoprotectants, and glass forming agents remain in the same phase with the protein in order to impart stability. Sugars, polymers, and polyols fall into this category and can sometimes serve all three roles.

Polyols encompass a class of excipients that includes sugars (e.g., mannitol, sucrose, or sorbitol), and other polyhydric alcohols (e.g., glycerol and propylene glycol). The polymer polyethylene glycol (PEG) is included in this category. Polyols are commonly used as stabilizing excipients and/or isotonicity agents in both liquid and lyophilized parenteral protein formulations. Polyols can protect proteins from both physical and chemical degradation pathways.

Exemplary C₃-C₆ polyols include propylene glycol, glycerin (glycerol), threose, threitol, erythrose, erythritol, ribose, arabinose, arabitol, lyxose, maltitol, sorbitol, sorbose, glucose, mannose, mannitol, levulose, dextrose, maltose, trehalose, fructose, xylitol, inositol, galactose, xylose, fructose, sucrose, 1,2,6-hexanetriol and the like. Higher order sugars include dextran, propylene glycol, or polyethylene glycol. Reducing sugars such as fructose, maltose or galactose oxidize more readily than do non-reducing sugars. Additional examples of sugar alcohols are glucitol, maltitol, lactitol or iso-maltulose. Additional exemplary lyoprotectants include glycerin and gelatin, and the sugars mellibiose, melezitose, raffinose, mannotriose and stachyose. Examples of reducing sugars include glucose, maltose, lactose, maltulose, iso-maltulose and lactulose. Examples of non-reducing sugars include non-reducing glycosides of polyhydroxy compounds selected from sugar alcohols and other straight chain polyalcohols. Monoglycosides include compounds obtained by reduction of disaccharides such as lactose, maltose, lactulose and maltulose.

### Amino Acids

In some embodiments, the pharmaceutical compositions described herein further comprise one or more amino acids as buffers, bulking agents, stabilizers and/or antioxidants. Histidine and glutamic acid can be employed to buffer protein formulations in the pH range of pH 5.5 — pH 6.5 and pH 4.0 — pH 5.5 respectively. The amino acids glycine, proline, serine and alanine stabilize proteins.

In some embodiments, the formulation further comprises an amino acid other than histidine.

In some embodiments, the formulation further comprises arginine, optionally in an amount ranging from about 10 mM to about 250 mM (e.g., about 10 mM, about 20 mM, about 30 mM, about 40 mM, about 50 mM, about 60 mM, about 70 mM, about 80 mM, about 90 mM, about 100 mM, about 110 mM, about 120 mM, about 130 mM, about 140 mM, about 150 mM, about 160 mM, about 170 mM, about 180 mM, about 190 mM, about 200 mM, about 210 mM, about 220 mM, about 230 mM, about 240 mM or about 250 mM). In some embodiments, the formulation further comprises arginine in an amount of about 100 mM.

In some embodiments, the formulation further comprises methionine, optionally in an amount ranging from about 10 mM to about 100 mM (e.g., about 10 mM, about 20 mM, about 30 mM, about 40 mM, about 50 mM, about 60 mM, about 70 mM, about 80 mM, about 90 mM, or about 100 mM). In some embodiments, the formulation further comprises methionine in an amount of about 20 mM.

### Antioxidants

In some embodiments, the pharmaceutical composition described herein further comprises one or more antioxidants. Oxidation of protein residues arises from a number of different sources. Beyond the addition of specific antioxidants, the prevention of oxidative protein damage involves the careful control of a number of factors throughout the manufacturing process and storage of the product such as atmospheric oxygen, temperature, light exposure, and chemical contamination. The most commonly used pharmaceutical antioxidants are reducing agents, oxygen/free-radical scavengers, or chelating agents. Antioxidants in therapeutic protein formulations must be water-soluble and remain active throughout the product shelf-life. Reducing agents and oxygen/free-radical scavengers work by ablating active oxygen species in solution. Chelating agents such as EDTA can be effective by binding trace metal contaminants that promote free-radical formation.

However, antioxidants themselves can induce other covalent or physical changes to the protein. Selection of an appropriate antioxidant is made according to the specific stresses and sensitivities of the protein.

### Metal Ions

In some embodiments, the pharmaceutical composition further comprises one or more metal ions. In general, transition metal ions are undesired in protein formulations because they can catalyze physical and chemical degradation reactions in proteins. However, specific metal ions are included in formulations when they are co-factors to proteins and in suspension formulations of proteins where they form coordination complexes (e.g., zinc suspension of insulin).

### Preservatives

In some embodiments, the pharmaceutical composition further comprises one or more preservatives. Preservatives may be necessary when developing multi-use parenteral formulations that involve more than one extraction from the same container. Preservatives that my be used include phenol, benzyl alcohol, meta-cresol, alkyl parabens such as methyl paraben or propyl paraben, benzalkonium chloride, and benzethonium chloride. Other examples of compounds with antimicrobial preservative activity include octadecyldimethylbenzyl ammonium chloride, hexamethonium chloride. Other types of preservatives include aromatic alcohols such as butyl alcohol, phenol, benzyl alcohol; atechol, resorcinol, cyclohexanol, 3-pentanol.

Some preservatives can cause injection site reactions, which is another factor for consideration when choosing a preservative. However, the disclosure also contemplates a pharmaceutical composition that does not comprise any preservatives.

### Antibodies in the Formulation

An “anti-sclerostin antibody” or an “antibody that binds to sclerostin” is an antibody that binds to sclerostin of SEQ ID NO: 1 or portions thereof. Recombinant human sclerostin/SOST is commercially available from, e.g., R&D Systems (Minneapolis, Mn., USA; 2006 Catalog #1406-ST-025). U.S. Pat. Nos. 6,395,511 and 6,803,453, and U.S. Patent Publication Nos. 2004/0009535 and 2005/0106683 refer to anti-sclerostin antibodies generally. Examples of sclerostin antibodies suitable for use in the context of the invention also are described in U.S. Patent Publication Nos. 2007/0110747 and 2007/0072797, which are hereby incorporated by reference in their entireties. Additional information regarding materials and methods for generating sclerostin antibodies can be found in U.S. Patent Publication No. 20040158045 (hereby incorporated by reference).

The term “antibody” refers to an intact immunoglobulin molecule (including polyclonal, monoclonal, chimeric, humanized, and/or human versions having full length heavy and/or light chains).

“Specifically binds” as used herein means that the antibody preferentially binds the antigen over other proteins. In some embodiments, “specifically binds” means the antibody has a higher affinity for the antigen than for other proteins. Antibodies that specifically bind an antigen may have a binding affinity for the antigen of less than or equal to 1×10⁻⁷ M, less than or equal to 2×10⁻⁷ M, less than or equal to 3×10⁻⁷ M, less than or equal to 4×10⁻⁷ M, less than or equal to 5×10⁻⁷ M, less than or equal to 6×10⁻⁷ M, less than or equal to 7×10⁻⁷ M, less than or equal to 8×10⁻⁷ M, less than or equal to 9×10⁻⁷ M, less than or equal to 1×10⁻⁸ M, less than or equal to 2×10⁻⁸ M, less than or equal to 3×10⁻⁸ M, less than or equal to 4×10⁻⁸ M, less than or equal to 5×10⁻⁸ M, less than or equal to 6×10⁻⁸ M, less than or equal to 7×10⁻⁸ M, less than or equal to 8×10⁻⁸ M, less than or equal to 9×10⁻⁸ M, less than or equal to 1×10⁻⁹ M, less than or equal to 2×10⁻⁹ M, less than or equal to 3×10⁻⁹ M, less than or equal to 4×10⁻⁹ M, less than or equal to 5×10⁻⁹ M, less than or equal to 6×10⁻⁹ M, less than or equal to 7×10⁻⁹ M, less than or equal to 8×10⁻⁹ M, less than or equal to 9×10⁻⁹ M, less than or equal to 1×10⁻¹⁰ M, less than or equal to 2×10^{31 19} M, less than or equal to 3×10⁻¹⁹ M, less than or equal to 4×10⁻¹⁹ M, less than or equal to 5×10⁻¹⁹ M, less than or equal to 6×10⁻¹⁹ M, less than or equal to 7×10⁻¹⁹ M, less than or equal to 8×10⁻¹⁹ M, less than or equal to 9×10⁻¹⁹ M, less than or equal to 1×10⁻¹¹ M, less than or equal to 2×10⁻¹¹ M, less than or equal to 3×10⁻¹¹ M, less than or equal to 4×10⁻¹¹ M, less than or equal to 5×10⁻¹¹ M, less than or equal to 6×10⁻¹¹ M, less than or equal to 7×10⁻¹¹ M, less than or equal to 8×10⁻¹¹ M, less than or equal to 9×10⁻¹¹ M, less than or equal to 1×10⁻¹² M, less than or equal to 2×10⁻¹² M, less than or equal to 3×10⁻¹² M, less than or equal to 4×10⁻¹² M, less than or equal to 5×10⁻¹² M, less than or equal to 6×10⁻¹² M, less than or equal to 7×10⁻¹² M, less than or equal to 8×10⁻¹² M, or less than or equal to 9×10⁻¹² M.

In some or any embodiments, the antibody binds to sclerostin of SEQ ID NO: 1, or a naturally occurring variant thereof, with an affinity (Kd) of less than or equal to 1×10⁻⁷ M, less than or equal to 1×10⁻⁸ M, less than or equal to 1×10⁻⁹ M, less than or equal to 1×10⁻¹⁰ M, less than or equal to 1×10⁻¹¹ M, or less than or equal to 1×10⁻¹² M. Affinity is determined using a variety of techniques, an example of which is an affinity ELISA assay. In various embodiments, affinity is determined by a BlAcore assay. In various embodiments, affinity is determined by a kinetic method. In various embodiments, affinity is determined by an equilibrium/solution method. U.S. Patent Publication No. 2007/0110747 (the disclosure of which is incorporated herein by reference) contains additional description of affinity assays suitable for determining the affinity (Kd) of an antibody for sclerostin.

In some or any embodiments, the anti-sclerostin antibody described herein preferably modulates sclerostin function in the cell-based assay described in U.S. Patent Publication No. 2007/0110747 and/or the in vivo assay described in U.S. Patent Publication No. 20070110747 and/or bind to one or more of the epitopes described in U.S. Patent Publication No. 2007/0110747 and/or cross-block the binding of one of the antibodies described in U.S. Patent Publication No. 2007/0110747 and/or are cross-blocked from binding sclerostin by one of the antibodies described in U.S. Patent Publication No. 2007/0110747 (incorporated by reference in its entirety and for its description of assays for characterizing an anti-sclerostin antibody).

“CDR” refers to the complementarity determining region within antibody variable sequences. There are three CDRs in each of the variable regions of the heavy chain and the light chain, which are designated CDR1, CDR2 and CDR3, for each of the variable regions. The term “ set of six CDRs” as used herein refers to a group of three CDRs that occur in the light chain variable region and heavy chain variable region, which are capable of binding the antigen. The exact boundaries of CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Chothia and coworkers (Chothia & Lesk, J. Mol. Biol. 196:901-917 (1987) and Chothia et al., Nature 342:877-883 (1989)) found that certain sub-portions within Kabat CDRs adopt nearly identical peptide backbone conformations, despite having great diversity at the level of amino acid sequence. These sub-portions were designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (FASEB J. 9:133-139 (1995)) and MacCallum (J Mol Biol 262(5):73245 (1996)). Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although preferred embodiments use Kabat or Chothia defined CDRs.

CDRs are obtained by, e.g., constructing polynucleotides that encode the CDR of interest. Such polynucleotides are prepared, for example, by using the polymerase chain reaction to synthesize the variable region using mRNA of antibody-producing cells as a template (see, for example, Larrick et al., Methods: A Companion to Methods in Enzymology, 2:106 (1991); Courtenay-Luck, “Genetic Manipulation of Monoclonal Antibodies,” in Monoclonal Antibodies Production, Engineering and Clinical Application, Ritter et al. (eds.), page 166, Cambridge University Press (1995); and Ward et al., “Genetic Manipulation and Expression of Antibodies,” in Monoclonal Antibodies: Principles and Applications, Birch et al., (eds.), page 137, Wiley-Liss, Inc. (1995)).

In various aspects, the antibody comprises at least one CDR sequence having at least 75% identity (e.g., at least 75%, 80%, 85%, 90%, 95% or 100% identity) to a CDR selected from CDR-H1, CDR-H2, CDR-H3, CDR-L1, CDR-L2, and CDR-L3 wherein CDR-H1 has the sequence given in SEQ ID NO: 2, CDR-H2 has the sequence given in SEQ ID NO: 3, CDR-H3 has the sequence given in SEQ ID NO: 4, CDR-L1 has the sequence given in SEQ ID NO: 5, CDR-L2 has the sequence given in SEQ ID NO: 6 and CDR-L3 has the sequence given in SEQ ID NO: 7. The anti-sclerostin antibody, in various aspects, comprises two of the CDRs or six of the CDRs.

In a preferred embodiment, the anti-sclerostin antibody comprises a set of six CDRs as follows: CDR-H1 of SEQ ID NO: 2, CDR-H2 of SEQ ID NO: 3, CDR-H3 of SEQ ID NO: 4, CDR-L1 of SEQ ID NO: 5, CDR-L2 of SEQ ID NO: 6 and CDR-L3 of SEQ ID NO: 7.

In some or any embodiments, the antibody comprises a light chain variable region comprising an amino acid sequence having at least 75% identity (e.g., at least 75%, 80%, 85%, 90%, 95% or 100% identity) to the amino acid sequence set forth in SEQ ID NO: 8 and a heavy chain variable region comprising an amino acid sequence having at least 75% identity (e.g., at least 75%, 80%, 85%, 90%, 95% or 100% identity) to the amino acid sequence set forth in SEQ ID NO: 9. In various aspects, the difference in the sequence compared to SEQ ID NO: 8 or 9 lies outside the CDR region in the corresponding sequences. In some or any embodiments, the antibody comprises a light chain variable region comprising an amino acid sequence set forth in SEQ ID NO: 8 and a heavy chain variable region comprising an amino acid sequence set forth in SEQ ID NO: 9.

In some or any embodiments, the anti-sclerostin antibody comprises all or part of a heavy chain (e.g., two heavy chains) comprising an amino acid sequence having at least 75% identity (e.g., at least 75%, 80%, 85%, 90%, 95% or 100% identity) to the amino acid sequence set forth in SEQ ID NO: 11 and all or part of a light chain (e.g., two light chains) comprising an amino acid sequence having at least 75% identity (e.g., at least 75%, 80%, 85%, 90%, 95% or 100% identity) to the amino acid sequence set forth in SEQ ID NO 10.

In some or any embodiments, the anti-sclerostin antibody comprises all or part of a heavy chain (e.g., two heavy chains) comprising an amino acid sequence having at least 75% identity (e.g., at least 75%, 80%, 85%, 90%, 95% or 100% identity) to the amino acid sequence set forth in SEQ ID NO: 13 and all or part of a light chain (e.g., two light chains) comprising an amino acid sequence having at least 75% identity (e.g., at least 75%, 80%, 85%, 90%, 95% or 100% identity) to the amino acid sequence set forth in SEQ ID NO 12.

Examples of other anti-sclerostin antibodies include, but are not limited to, the anti-sclerostin antibodies disclosed in International Patent Publication Nos. WO 2008/092894, WO 2008/115732, WO 2009/056634, WO 2009/047356, WO 2010/100200, WO 2010/100179, WO 2010/115932, and WO 2010/130830 (each of which is incorporated by reference herein in its entirety).

It will be understood by one skilled in the art that some proteins, such as antibodies, may undergo a variety of posttranslational modifications. The type and extent of these modifications often depends on the host cell line used to express the protein as well as the culture conditions. Such modifications may include variations in glycosylation, methionine oxidation, diketopiperizine formation, aspartate isomerization and asparagine deamidation. A frequent modification is the loss of a carboxy-terminal basic residue (such as lysine or arginine) due to the action of carboxypeptidases (as described in Harris, R J. Journal of Chromatography 705:129-134, 1995).

Other modifications include hydroxylation of proline and lysine, phosphorylation of hydroxyl groups of seryl or threonyl residues, methylation of the a-amino groups of lysine, arginine, and histidine side chains (T. E. Creighton, Proteins: Structure and Molecular Properties, W. H. Freeman & Co., San Francisco, pp. 79-86 [1983], entirely incorporated by reference), acetylation of the N-terminal amine, and amidation of any C-terminal carboxyl group.

In some embodiments, the anti-sclerostin antibody in the formulation is present at a concentration of at least about 70 mg/ml, about 71 mg/ml, about 72 mg/ml, about 73 mg/ml, about 74 mg/ml, about 75 mg/ml, about 76 mg/ml, about 77 mg/ml, about 78 mg/ml, about 79 mg/ml, about 80 mg/ml, about 81 mg/ml, about 82 mg/ml, about 83 mg/ml, about 84 mg/ml, about 85 mg/ml, about 86 mg/ml, about 87 mg/ml, about 88 mg/ml, about 89 mg/ml, about 90 mg/ml, about 91 mg/ml, about 92 mg/ml, about 93 mg/ml, about 94 mg/ml, about 95 mg/ml, about 96 mg/ml, about 97 mg/ml, about 98 mg/ml, about 99 mg/ml, about 100 mg/ml, about 101 mg/ml, about 102 mg/ml, about 103 mg/ml, about 104 mg/ml, about 105 mg/ml, about 106 mg/ml, about 107 mg/ml, about 108 mg/ml, about 109 mg/ml, about 110 mg/ml, about 111 mg/ml, about 112 mg/ml, about 113 mg/ml, about 114 mg/ml, about 115 mg/ml, about 116 mg/ml, about 117 mg/ml, about 118 mg/ml, about 119 mg/ml, about 120 mg/ml, about 121 mg/ml, about 122 mg/ml, about 123 mg/ml, about 124 mg/ml, about 125 mg/ml, about 126 mg/ml, about 127 mg/ml, about 128 mg/ml, about 129 mg/ml, about 130 mg/ml, about 131 mg/ml, about 132 mg/ml, about 132 mg/ml, about 133 mg/ml, about 134 mg/ml, about 135 mg/ml, about 136 mg/ml, about 137 mg/ml, about 138 mg/ml, about 139 mg/ml, about 140 mg/ml, about 141 mg/ml, about 142 mg/ml, about 143 mg/ml, about 144 mg/ml, about 145 mg/ml, about 146 mg/ml, about 147 mg/ml, about 148 mg/ml, about 149 mg/ml, about 150 mg/ml, about 151 mg/ml, about 152 mg/ml, about 153 mg/ml, about 154 mg/ml, about 155 mg/ml, about 156 mg/ml, about 157 mg/ml, about 158 mg/ml, about 159 mg/ml, or about 160 mg/ml, and may range up to , e.g., about 300 mg/ml, about 290 mg/ml, about 280 mg/ml, about 270 mg/ml, about 260 mg/ml, about 250 mg/ml, about 240 mg/ml, about 230 mg/ml, about 220 mg/ml, about 210 mg/ml, about 200 mg/ml, about 190 mg/ml, about 180 mg/ml, or about 170 mg/ml. Any range featuring a combination of the foregoing endpoints is contemplated, including but not limited to: about 70 mg/ml to about 250 mg/ml, about 70 mg/ml to about 200 mg/ml, about 70 mg/ml to about 160 mg/ml, about 100 mg/ml to about 250 mg/ml, about 100 mg/I to about 200 mg/ml, or about 100 mg/ml to about 180 mg/ml.

### Viscosity

In some embodiments, the viscosity of a composition comprising one or more of the antibodies described herein is determined. The term “viscosity” as used herein refers to “absolute viscosity.” Absolute viscosity, sometimes called dynamic or simple viscosity, is the product of kinematic viscosity and fluid density (Absolute Viscosity=Kinematic Viscosity×Density). The dimension of kinematic viscosity is L²/T where L is a length and T is a time. Commonly, kinematic viscosity is expressed in centistokes (cSt). The SI unit of kinematic viscosity is mm²/s, which is 1 cSt. Absolute viscosity is expressed in units of centipoise (cP). The SI unit of absolute viscosity is the millipascal-second (mPa-s), where 1 cP=1 mPa-s.

The viscosity of a composition can be measured hours (e.g., 1-23 hours), days (e.g., 1-10 days), weeks (e.g., 1-5 weeks), months (e.g., 1-12 months), or years (e.g., 1-2 years, 1-3 years) after the addition of the antibody to the composition. Viscosity measurements may be made at a storage or administration temperature, e.g. 2-8° C. or 25° C. (room temperature). In some embodiments, absolute viscosity of the liquid or reconstituted liquid composition at the storage and/or administration temperature is 15 cP or less, or 14, 13, 12, 11, 10, 9, 8, 7, 6, 5, or 4 cP or less. In some embodiments, absolute viscosity of the liquid or reconstituted liquid composition is 6 cP or less.

In some embodiments, the viscosity of the antibody composition is measured prior to and after the addition of antibody. Methods of measuring viscosity are well known in the art and include, for example, using a capillary viscometer, or a cone-plate rheometer. Any method may be used provided the same method is used to compare the test and reference formulations.

### Therapeutic Methods

The antibody and pharmaceutical compositions described herein are useful for treating or preventing bone-related disorders, such as bone-related disorders associated with abnormal osteoblast or osteoclast activity. In some embodiments, the antibody is administered to a subject suffering from a bone related disorder selected from the group consisting of achondroplasia, cleidocranial dysostosis, enchondromatosis, fibrous dysplasia, Gaucher's Disease, hypophosphatemic rickets, Marfan's syndrome, multiple hereditary exotoses, neurofibromatosis, osteogenesis imperfecta, osteopetrosis, osteopoikilosis, sclerotic lesions, pseudoarthrosis, pyogenic osteomyelitis, periodontal disease, anti-epileptic drug induced bone loss, primary and secondary hyperparathyroidism, familial hyperparathyroidism syndromes, weightlessness induced bone loss, osteoporosis in men, postmenopausal bone loss, osteoarthritis, renal osteodystrophy, infiltrative disorders of bone, oral bone loss, osteonecrosis of the jaw, juvenile Paget's disease, melorheostosis, metabolic bone diseases, mastocytosis, sickle cell anemia/disease, organ transplant related bone loss, kidney transplant related bone loss, systemic lupus erythematosus, ankylosing spondylitis, epilepsy, juvenile arthritides, thalassemia, mucopolysaccharidoses, Fabry Disease, Turner Syndrome, Down Syndrome, Klinefelter Syndrome, leprosy, Perthe's Disease, adolescent idiopathic scoliosis, infantile onset multi-system inflammatory disease, Winchester Syndrome, Menkes Disease, Wilson's Disease, ischemic bone disease (such as Legg-Calve-Perthes disease and regional migratory osteoporosis), anemic states, conditions caused by steroids, glucocorticoid-induced bone loss, heparin-induced bone loss, bone marrow disorders, scurvy, malnutrition, calcium deficiency, osteoporosis, osteopenia, alcoholism, chronic liver disease, postmenopausal state, chronic inflammatory conditions, rheumatoid arthritis, inflammatory bowel disease, ulcerative colitis, inflammatory colitis, Crohn's disease, oligomenorrhea, amenorrhea, pregnancy-related bone loss, diabetes mellitus, hyperthyroidism, thyroid disorders, parathyroid disorders, Cushing's disease, acromegaly, hypogonadism, immobilization or disuse, reflex sympathetic dystrophy syndrome, regional osteoporosis, osteomalacia, bone loss associated with joint replacement, HIV associated bone loss, bone loss associated with loss of growth hormone, bone loss associated with cystic fibrosis, chemotherapy-associated bone loss, tumor-induced bone loss, cancer-related bone loss, hormone ablative bone loss, multiple myeloma, drug-induced bone loss, anorexia nervosa, disease-associated facial bone loss, disease-associated cranial bone loss, disease-associated bone loss of the jaw, disease-associated bone loss of the skull, bone loss associated with aging, facial bone loss associated with aging, cranial bone loss associated with aging, jaw bone loss associated with aging, skull bone loss associated with aging, and bone loss associated with space travel.

In some embodiments, the antibodies described herein are useful for improving outcomes in orthopedic procedures, dental procedures, implant surgery, joint replacement, bone grafting, bone cosmetic surgery and bone repair such as fracture healing, nonunion healing, delayed union healing and facial reconstruction. A composition comprising one or more antibodies may be administered before, during and/or after the procedure, replacement, graft, surgery or repair.

In some embodiments, the antibodies described herein are useful for the treatment of any fracture comprising a gap between two segments of bone (e.g., a gap of at least about 1 mm between two segments of bone). In some or any embodiments, the gap is at least about 2 mm, at least about 3 mm, at least about 4 mm, at least about 5 mm, at least about 6 mm, at least about 7 mm, at least about 8 mm, at least about 9 mm, or at least about 1 cm or more. In some or any embodiments, the gap is about 5 mm to 1 cm, or up to 1 cm. The terms “bone gap defect” and “segmental skeletal defect” are used synonymously herein and refer to a gap between two segments of bone (e.g., a gap of at least 1 mm).

Exemplary bone gap defects include, but are not limited to, a comminuted fracture, a non-union fracture, a segmental skeletal defect, surgically created bone defects, surgically treated bone defects, and bone defects created from traumatic injury to the bone or disease (including, but not limited to, arthritis, tumor removal (resection) or infection removal). In some or any embodiments, the bone gap defect is produced by removal of infected sections of bone or the removal of cancer from the bone due to bone cancers including, but not limited to, osteosarcoma, Ewing's sarcoma, chondrosarcoma, malignant fibrous histiocytoma, fibrosarcoma, and chordoma. In some or any embodiments, the bone gap defect is a developmental deformity, e.g., due to a genetic defect.

In some or any embodiments, the bone gap defect is produced by removal of sections of bone containing a benign tumor. Exemplary benign bone tumors include, but are not limited to, osteoma, osteoid osteoma, osteoblastoma, osteochondroma, enchondroma, chonrdomyxoid fibroma, aneurysmal bone cyst, unicameral bone cyst, fibrous dysplasia of bone and giant cell tumor of the bone.

The antibody need not cure the subject of the disorder or completely protect against the onset of a bone-related disorder to achieve a beneficial biological response. The antibody may be used prophylactically, meaning to protect, in whole or in part, against a bone-related disorder or symptom thereof. The antibody also may be used therapeutically to ameliorate, in whole or in part, a bone-related disorder or symptom thereof, or to protect, in whole or in part, against further progression of a bone-related disorder or symptom thereof. Indeed, the materials and methods of the invention are particularly useful for increasing bone mineral density, and optionally maintaining the increased bone mineral density over a period of time.

In some embodiments, one or more administrations of an antibody described herein are carried out over a therapeutic period of, for example, about 1 week to about 18 months (e.g., about 1 month to about 12 months, about 1 month to about 9 months or about 1 month to about 6 months or about 1 month to about 3 months). In some embodiments, a subject is administered one or more doses of a antibody described herein over a therapeutic period of, for example about 1 month to about 12 months (52 weeks) (e.g., about 2 months, about 3 months, about 4 months, about 5 months, about 6 months, about 7 months, about 8 months, about 9 months, about 10 months, or about 11 months).

In addition, it may be advantageous to administer multiple doses of the antibody or space out the administration of doses, depending on the therapeutic regimen selected for a particular subject. In some embodiments, the antibody or fragment thereof is administered periodically over a time period of one year (12 months, 52 weeks) or less (e.g., 9 months or less, 6 months or less, or 3 months or less). In this regard, the antibody or fragment thereof is administered to the human once every about 3 days, or about 7 days, or 2 weeks, or 3 weeks, or 4 weeks, or 5 weeks, or 6 weeks, or 7 weeks, or 8 weeks, or 9 weeks, or 10 weeks, or 11 weeks, or 12 weeks, or 13 weeks, or 14 weeks, or 15 weeks, or 16 weeks, or 17 weeks, or 18 weeks, or 19 weeks, or 20 weeks, or 21 weeks, or 22 weeks, or 23 weeks, or 6 months, or 12 months.

In some embodiments, one or more doses of the antibody are administered in an amount and for a time effective to increase bone mineral density or treat a bone disorder associated with decreased bone mineral density. In various embodiments, one or more doses comprising from about 50 milligrams to about 1,000 milligrams of the antibody are administered per week to a subject (e.g., a human subject). For example, a dose of antibody can comprise at least about 5 mg, 15 mg, 25 mg, 50 mg, about 60 mg, about 70 mg, about 80 mg, about 90 mg, about 100 mg, about 120 mg, about 150 mg, about 200 mg, about 210 mg, about 240 mg, about 250 mg, about 280 mg, about 300 mg, about 350 mg, about 400 mg, about 420 mg, about 450 mg, about 500 mg, about 550 mg, about 600 mg, about 650 mg, about 700 mg, about 750 mg, about 800 mg, about 850 mg, about 900 mg, about 950 mg or up to about 1,000 mg of antibody. Ranges between any and all of these endpoints are also contemplated, e.g. about 50 mg to about 80 mg, about 70 mg to about 140 mg, about 70 mg to about 270 mg, about 75 mg to about 100 mg, about 100 mg to about 150 mg, about 140 mg to about 210 mg, or about 150 mg to about 200 mg, or about 180 mg to about 270 mg, or about 280 to about 410 mg. The dose is administered at any interval, such as multiple times a week (e.g., twice or three times per week), once a week, once every two weeks, once every three weeks, or once every four weeks. In some or any embodiments, a dose of antibody ranging from about 120 mg to about 210 mg is administered twice a month. In some or any embodiments, a dose of about 140 mg of the antibody is administered twice a month. In various aspects, a dose of about 210 mg of antibody is administered once a month.

In some embodiments, the one or more doses of antibody can comprise between about 0.1 to about 50 milligrams (e.g., between about 5 and about 50 milligrams), or about 1 to about 100 milligrams, of antibody per kilogram of body weight (mg/kg). For example, the dose of antibody may comprise at least about 0.1 mg/kg, about 0.5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 20 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, or about 49 mg/kg, or about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, or up to about 100 mg/kg. Ranges between any and all of these endpoints are also contemplated, e.g., about 1 mg/kg to about 3 mg/kg, about 1 mg/kg to about 5 mg/kg, about 1 mg/kg to about 8 mg/kb, about 3 mg/kg to about 8 mg.kg, about 1 mg/kg to about 10 mg/kg, about 1 mg/kg to about 20 mg/kg, about 1 mg/kg to about 40 mg/kg, about 5 mg/kg to about 30 mg/kg, or about 5 mg/kg to about 20 mg/kg.

### Monitoring Therapy

Antibody-mediated increases in bone mineral content or bone density may be measured using single- and dual-energy X-ray absorptometry, ultrasound, computed tomography, radiography, and magnetic resonance imaging. The amount of bone mass may also be calculated from body weights or by using other methods (see Guinness-Hey, Metab. Bone Dis. Relat. Res., 5:177-181 (1984)). Animal models are used in the art for testing the effect of the pharmaceutical compositions and methods on, for example, parameters of bone loss, bone resorption, bone formation, bone strength, or bone mineralization that mimic conditions of human disease such as osteoporosis and osteopenia. Examples of such models include the ovariectomized rat model (Kalu, Bone and Mineral, 15:175-192 (1991); Frost and Jee, Bone and Mineral, 18:227-236 (1992); and Jee and Yao, J. Musculoskel. Neuron. Interact., 1:193-207 (2001)). The methods for measuring antibody activity described herein also may be used to determine the efficacy of other sclerostin inhibitors.

In humans, bone mineral density can be determined clinically using dual x-ray absorptiometry (DXA) of, for example, the hip and spine. Other techniques include quantitative computed tomography (QCT), ultrasonography, single-energy x-ray absorptiometry (SXA), and radiographic absorptiometry. Common central skeletal sites for measurement include the spine and hip; peripheral sites include the forearm, finger, wrist and heel. Except for ultrasonography, the American Medical Association notes that BMD techniques typically involve the use of x-rays and are based on the principle that attenuation of the radiation depends on thickness and composition of the tissues in the radiation path. All techniques involve the comparison of results to a normative database.

Alternatively, a physiological response to one or more anti-sclerostin antibodies can be gauged by monitoring bone marker levels. Bone markers are products created during the bone remodeling process and are released by bone, osteoblasts, and/or osteoclasts. Fluctuations in bone resorption and/or bone formation “marker” levels imply changes in bone remodeling/modeling. The International Osteoporosis Foundation (IOF) recommends using bone markers to monitor bone density therapies (see, e.g., Delmas et al., Osteoporos Int., Suppl. 6:S2-17 (2000), incorporated herein by reference). Markers indicative of bone resorption (or osteoclast activity) include, for example, C-telopeptide (e.g., C-terminal telopeptide of type 1 collagen (CTX) or serum cross-linked C-telopeptide), N-telopeptide (N-terminal telopeptide of type 1 collagen (NTX)), deoxypyridinoline (DPD), pyridinoline, urinary hydroxyproline, galactosyl hydroxylysine, and tartrate-resistant acid phosphatase (e.g., serum tartrate-resistant acid phosphatase isoform 5b). Bone formation/mineralization markers include, but are not limited to, bone-specific alkaline phosphatase (BSAP), peptides released from N- and C-terminal extension of type I procollagen (P1NP, PICP), and osteocalcin (OstCa). Several kits are commercially-available to detect and quantify markers in clinical samples, such as urine and blood.

### Combination Therapy

Treatment of a pathology by combining two or more agents that target the same pathogen or biochemical pathway or biological process sometimes results in greater efficacy and diminished side effects relative to the use of a therapeutically relevant dose of each agent alone. In some cases, the efficacy of the drug combination is additive (the efficacy of the combination is approximately equal to the sum of the effects of each drug alone), but in other cases the effect is synergistic (the efficacy of the combination is greater than the sum of the effects of each drug given alone). As used herein, the term “combination therapy” means that two or more agents are delivered in a simultaneous manner, e.g., concurrently, or wherein one of the agents is administered first, followed by the second agent, e.g., sequentially.

In some embodiments, the antibody is administered along with a standard of care therapeutic for the treatment of decreased bone mineral density (i.e., the antibody and standard of care therapeutic are part of the same treatment plan). As used herein, the term “standard of care” refers to a treatment that is generally accepted by clinicians for a certain type of patient diagnosed with a type of illness. In some embodiments, the antibody is administered along with a second bone-enhancing agent useful for the treatment of decreased bone mineral density or bone defect. In some embodiments, the bone-enhancing agent is selected from the group consisting of an anti-resorptive agent, a bone-forming agent (i.e., anabolic), an estrogen receptor modulator (including, but not limited to, raloxifene, bazedoxifene and lasofoxifene) and a drug that has an inhibitory effect on osteoclasts. In some embodiments, the second bone-enhancing agent is selected from the group consisting of a bisphosphonate (including, but not limited to, alendronate sodium (FOSAMAX®), risedronate, ibandronate sodium (BONIVA®) and zoledronic acid (RECLAST®)); an estrogen or estrogen analogue; an anti-RANK ligand (RANKL) inhibitor, such as an anti-RANKL antibody (e.g., denosumab, PROLIA®); vitamin D, or a vitamin D derivative or mimic thereof; a calcium source, a cathepsin-K (cat-K) inhibitor (e.g. odanacatib), Tibolone, calcitonin or a calcitriol; and hormone replacement therapy. In some embodiments, the second bone-enhancing agent includes, but is not limited to, parathyroid hormone (PTH) or a peptide fragment thereof, PTH-related protein (PTHrp), bone morphogenetic protein, osteogenin, NaF, a PGE2 agonist, a statin, strontium ranelate, and a sclerostin inhibitor (e.g., an anti-sclerostin antibody described in, for example, U.S. Pat. Nos. 7,592,429 or 7,872,106). In some embodiments, the second bone-enhancing agent is Tymlos® (abaloparatide), Forteo® (Teriparatide), Preotact®, or Protelos®. In some embodiments, the second bone-enhancing agent comprises a bone morphogenetic protein (e.g., BMP-1, BMP-2, BMP-3, BMP-4, BMP-5, BMP-6, BMP-7, BMP-8, BMP-9, BMP-10, BMP-11, BMP-12, BMP-13, BMP-14 and/or BMP-15).

In some embodiments, the combination therapy employing an antibody described herein may precede or follow administration of additional therapeutic(s) (e.g., second bone-enhancing agent) by intervals ranging from minutes to weeks to months. For example, separate modalities are administered within about 24 hours of each other, e.g., within about 6-12 hours of each other, or within about 1-2 hours of each other, or within about 10-30 minutes of each other. In some situations, it may be desirable to extend the time period for treatment significantly, where several days (2, 3, 4, 5, 6 or 7 days) to several weeks (1, 2, 3, 4, 5, 6, 7 or 8 weeks) lapse between the respective administrations of different modalities. Repeated treatments with one or both agents/therapies of the combination therapy is specifically contemplated.

### Kits

A pharmaceutical composition comprising one or more antibodies described herein may be placed within containers (e.g., vials or syringes), along with packaging material that provides instructions regarding the use of such pharmaceutical compositions. Generally, such instructions will include a tangible expression describing the antibody concentration, as well as within certain embodiments, relative amounts of excipient ingredients or diluents (e.g., water, saline or PBS) that may be necessary to reconstitute the pharmaceutical composition.

### EXAMPLES

### Example 1

### Stability Assessment

Samples were filled at 1 mL in 3 cc vials, for both protein and placebos. Romosozumab (70 mg/ml) was dialyzed into the formulation buffers identified below in Table 2, sterile filtered, and then filled under aseptic conditions. Storage temperatures were −70° C., −30° C., 4° C., 25° C., 37° C., and 45° C. Samples were stored for up to 24 months, pulled at specified time points and analyzed. Samples stored at accelerated temperatures, 25° C., 37° C., and 45° C., were stored for four weeks.

TABLE 2
Romosozumab formulations assessed

| Formulation | Buffer | Excipient | pH |
|---|---|---|---|
| 1 | 50 mM Na Acetate | 4% Sorbitol | 4.0 |
| 2 | 20 mM Na Acetate | 5% Sorbitol | 4.5 |
| 3 | 10 mM Na Acetate | 5% Sorbitol | 5.2 |
| 4 | 10 mM Na Acetate | 9% Sucrose | 5.2 |
| 5 | 10 mM Na Acetate | 5% Sorbitol | 5.8 |
| 6 | 10 mM Glutamic Acid | 5% Sorbitol | 4.5 |
| 7 | 10 mM Glutamic Acid | 5% Sorbitol | 5.2 |
| 8 | 10 mM Succinic Acid | 5% Sorbitol | 5.2 |
| 9 | 10 mM Histidine | 5% Sorbitol | 6.0 |

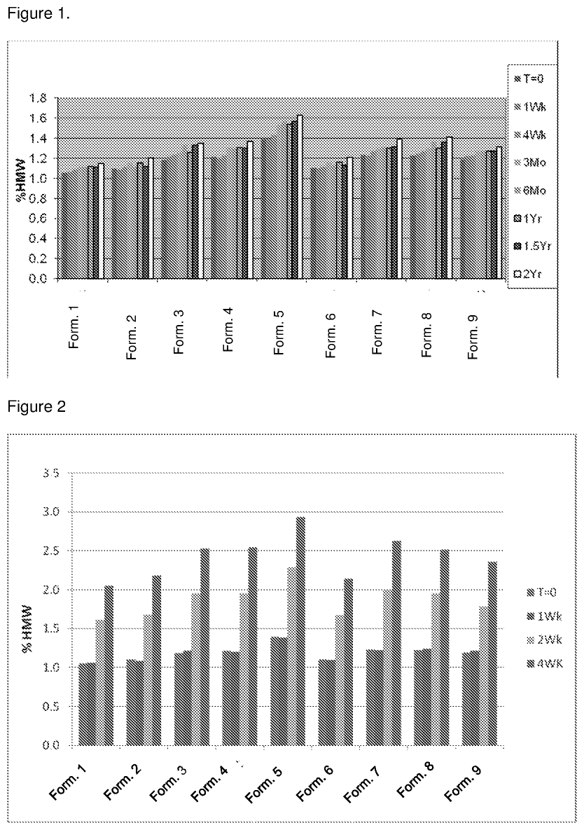

After storage at 4° C. for 24 months, Formulation 5 performed the poorest out of the panel when measured by SE-HPLC analysis of high-molecular weight species (FIG. 1). Results identified Formulations 1, 2 and 6 as generating the least amount of HMW (dimer) forms after 2 years of storage at 4° C. A similar stability profile is shown in for samples stored at −30° C. and −70° C. (data not shown) for 24 months.

All of the formulations studied fell within a range of 0.5% main peak as measured by peak area integration (data not shown). When stored at temperatures below 0° C., pH data over the 2 years show a similar trend to the 4° C. data, but not as marked (data not shown).

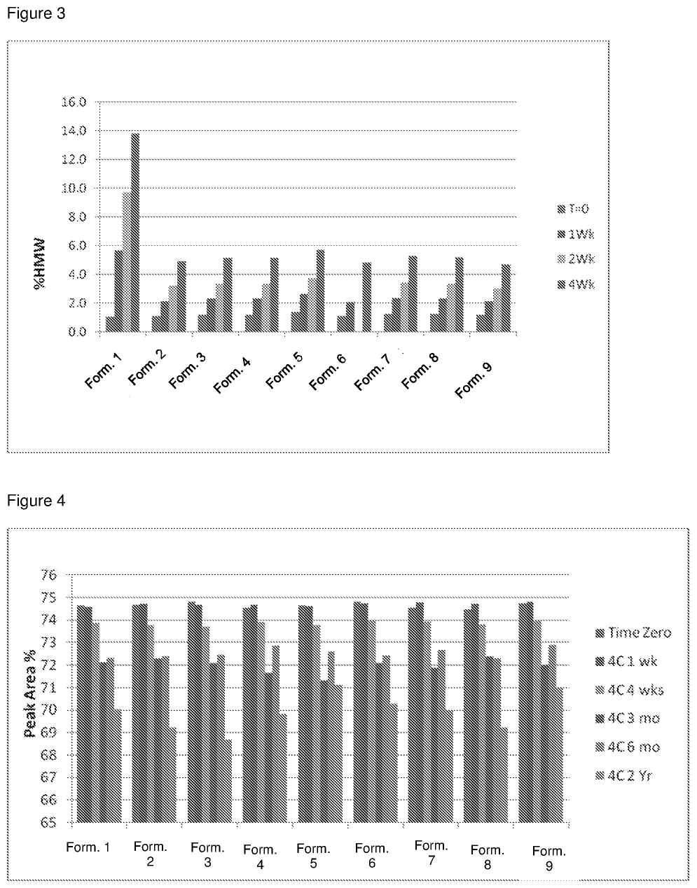

Accelerated temperature storage at 37° C. reveals a similar stability profile over four weeks of storage, though the percentages of HMW species are greater than at lower storage temperatures. Percent HMW of romosozumab increased the most significantly in Formulation 1 for samples stored at 45° C. (FIGS. 2 and 3).

Trends in sub-visible particles were determined by light-obscuration sub-visible particle detection (HIAC) to be similar for both romosozumab containing samples and placebo samples (data not shown). All particle counts are below compendial assay limits, though it is noted that romosozumab in Formulations 5, 7, 8 and 9 generated detectable levels throughout the stability period, while the last time point measured, 2 years (24 months), did not have the same levels of sub-visible particles at either 10 μM or 25 μM. In the placebo samples, Formulation 1 showed the highest level of sub-visible particles after 2 years, but still within USP limits for size and container (data not shown).

### Cation-Exchange HPLC

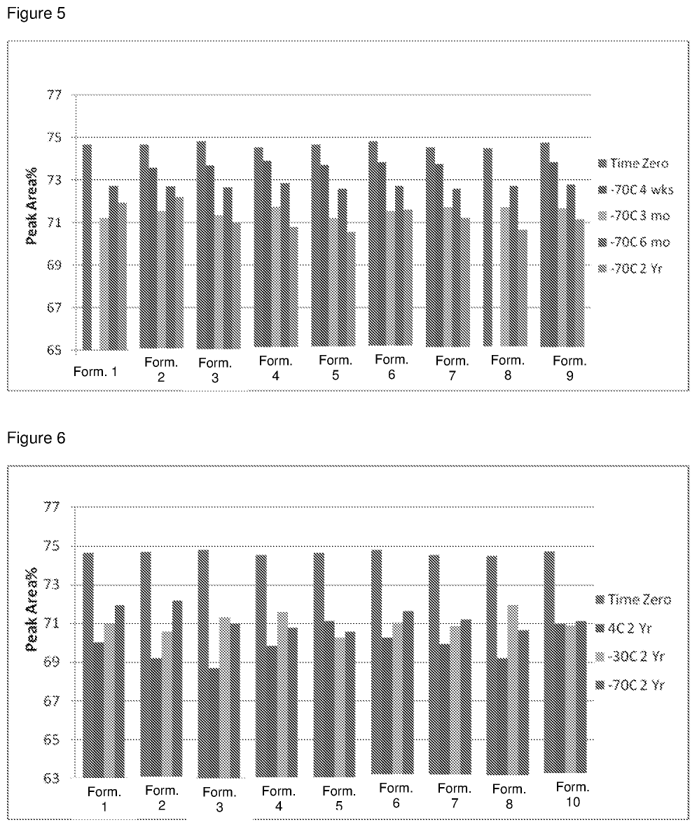

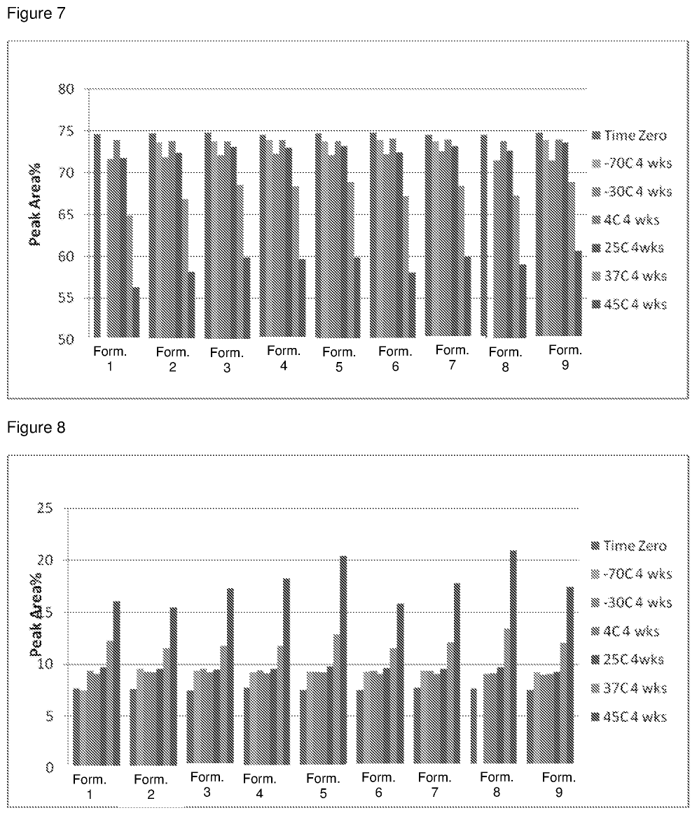

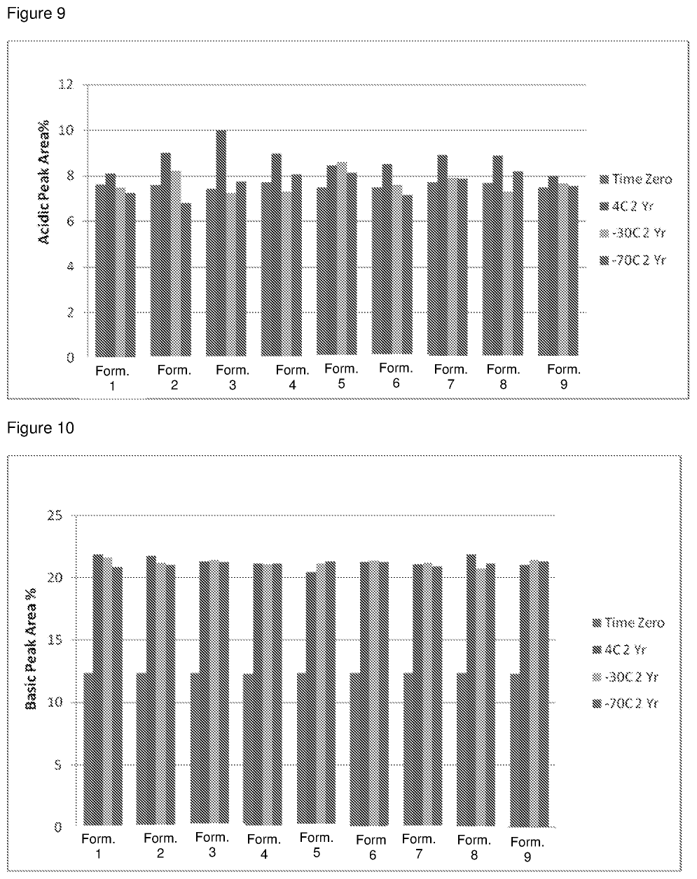

Long-term romosozumab main peak stability data at 4° C. and −70° C. are shown in FIGS. 4-10. Generally, both temperatures exhibit similar stability based on cation-exchange HPLC. Across temperatures and time, the histidine formulation, H6S, performed the most consistently (FIG. 6 for comparison of main peak data, FIG. 9 for acidic peak data). Comparing formulations by individual temperature storage out to 2 years, provide more detail with the inclusion of additional timepoints and are shown in FIGS. 4 and 5. Romosozumab stored at accelerated temperatures, which included 25° C., 37° C. and 45° C., show some different trends; comparing the acetate containing formulations stored at 4° C. with the 25° C., 37° C., and 45° C. main peak data (FIGS. 4 and 7). The main peak trends are reversed. The short-term stability trends agree, but diverge when the higher temperature results are compared to the long-term stability numbers.

### Example 2

### pH and Solubility Studies

Nine formulations were acetate based and eight were glutamate, histidine, or succinate buffered formulations. Isotonic amounts of excipients were used singly or in combination: glycerol, sucrose, arginine, and methionine. All formulations were prepared by dialyzing romosozumab into each formulation as listed in Table 3.

Each formulation tested contained 70 mg/mL romosozumab. Fills were 0.5 mL in 3 cc vials. Vialed samples were stored at −70° C., −30° C., 4° C., 25° C., 37° C., and 45° C. Samples were analyzed at set relevant time points by SEC-HPLC, CEX-HPLC, reduced CE-SDS, HIAC and both reduced and non-reduced SDS-PAGE.

Samples stored at accelerated temperatures were analyzed at 2 weeks, 4 weeks, 8 weeks, and 3-month time points. Samples stored at all other temperatures were analyzed at time points extending to two years.

TABLE 3
Formulations assessed

| Formulation | Buffer | Excipient | pH |
|---|---|---|---|
| 10 | 10 mM acetate | Glycerol 2.5% (v/v) | 4.8 |
| 11 | 10 mM acetate | Glycerol 2.5% (v/v) | 5.2 |
| 12 | 10 mM acetate | Glycerol 2.5% (v/v) | 5.6 |
| 13 | 30 mM glutamate | Glycerol 2.5% (v/v) | 5.2 |
| 14 | 10 mM histidine | Glycerol 2.5% (v/v) | 5.2 |
| 15 | 10 mM histidine | Glycerol 2.5% (v/v) | 5.6 |
| 16 | 10 mM succinate | Glycerol 2.5% (v/v) | 5.2 |
| 17 | 10 mM acetate | Sucrose 9% (w/v) | 4.8 |
| 18 | 10 mM acetate | Sucrose 9% (w/v) | 5.2 |
| 19 | 10 mM acetate | Sucrose 9% (w/v) | 5.6 |
| 20 | 30 mM glutamate | Sucrose 9% (w/v) | 5.2 |
| 21 | 10 mM histidine | Sucrose 9% (w/v) | 5.2 |
| 22 | 10 mM histidine | Sucrose 9% (w/v) | 5.6 |
| 23 | 10 mM succinate | Sucrose 9% (w/v) | 5.2 |
| 25 | 10 mM acetate | Glycerol 1% (v/v), | 5.2 |
| | | Arginine 100 mM | |
| 26 | 10 mM acetate | Glycerol 1% (v/v), | 5.6 |
| | | Arginine 100 mM | |
| 27 | 10 mM acetate | Glycerol 1% (v/v), | 5.2 |
| | | Arginine 100 mM | |
| 28 | 10 mM acetate | Sucrose 8.5% (w/v), | 5.2 |
| | | Methionine 20 mM | |

HIAC analysis performed after two years storage measured particle counts within USP guidelines for 10 and 25 micrometer sized particles (data not shown).

Arginine formulation 26 appeared turbid after both five and ten cycles of freeze-thaw at both −30° C. and −70° C. (data not shown). Particles were not analyzed for these samples due to that turbidity. All other formulations and placebos stored below 0° C. had particle counts below USP guidelines for 10 and 25 micrometer sized particles (data not shown). All samples stored at 4° C. at the 2-year time point were well under USP guideline limits (data not shown). Consistently across formulations studied, 3-month time point samples showed an increase of particles though this does not trend for the later time points.

### Size-Exclusion HPLC

High-molecular weight species increased generally with increased pH. Based on the SE-HPLC data, Formulations 17, 25, 26,and 28 performed similarly in suppressing HMW species formation at 4° C. Arginine-containing formulations suppressed high-molecular weight forms at accelerated temperatures. Tables 4 and 5 below provide the results of romosozumab in various formulations when stored at −30° C. and −70° C., respectively, at various time points (t0, 4 weeks, 3 months, 6 months, 1 year, 1.5 years and 2 years).

TABLE 4
% Main peak of romosozumab when
stored at −30° C. as assessed by SEC.

| Formulation | 0 | 4 W | 3 M | 6 M | 1 Yr | 1.5 Yr | 2 Yr |
|---|---|---|---|---|---|---|---|
| 13 | 97.8 | 97.8 | 97.6 | 97.3 | 97.8 | 96.9 | 97.0 |
| 29 | 97.8 | 97.9 | 97.8 | 97.3 | 97.9 | 96.8 | 96.9 |
| 14 | 97.8 | 97.7 | 97.6 | 97.2 | 97.8 | 96.9 | 97.0 |
| 15 | 97.7 | 97.7 | 97.4 | 97.2 | 97.7 | 96.8 | 97.0 |
| 21 | 97.8 | 97.8 | 97.5 | 97.3 | 97.8 | 96.7 | 97.0 |
| 22 | 97.7 | 97.7 | 97.5 | 97.3 | 97.6 | 96.6 | 96.8 |
| 16 | 97.7 | 97.7 | 97.5 | 97.2 | 97.6 | 96.9 | 97.0 |
| 23 | 97.7 | 97.8 | | 97.2 | 97.7 | | 96.7 |

TABLE 5
% Main peak of romosozumab when
stored at −70° C. as assessed by SEC.

| Formulation | 0 | 4 W | 3 M | 6 M | 1 Yr | 1.5 Yr | 2 Yr |
|---|---|---|---|---|---|---|---|
| 13 | 97.8 | 97.7 | 97.7 | 97.4 | 97.9 | 96.9 | 97.1 |
| 29 | 97.8 | 97.9 | 97.7 | 97.4 | 97.7 | 96.8 | 97.0 |
| 14 | 97.8 | 97.7 | 97.6 | 97.3 | 97.6 | 97.0 | 97.0 |
| 15 | 97.7 | 97.7 | 97.6 | 97.2 | 97.6 | 97.0 | 97.0 |
| 21 | 97.8 | 97.8 | 97.7 | 97.3 | 97.7 | 96.7 | 96.8 |
| 22 | 97.7 | 97.7 | 97.6 | 97.3 | 97.6 | 96.9 | 96.8 |
| 16 | 97.7 | 97.8 | 97.6 | 97.3 | 97.7 | 96.9 | 97.0 |
| 23 | 97.7 | | | 97.2 | 97.7 | | 96.8 |

### Cation-Exchange HPLC

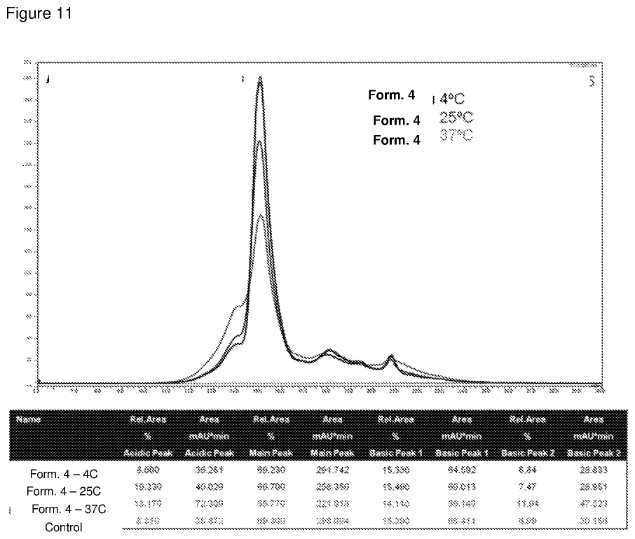

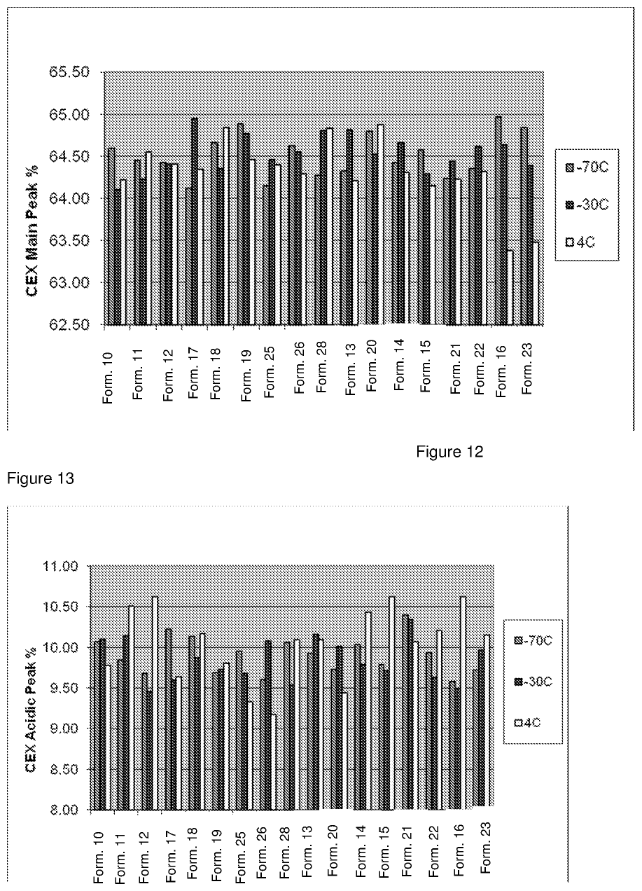

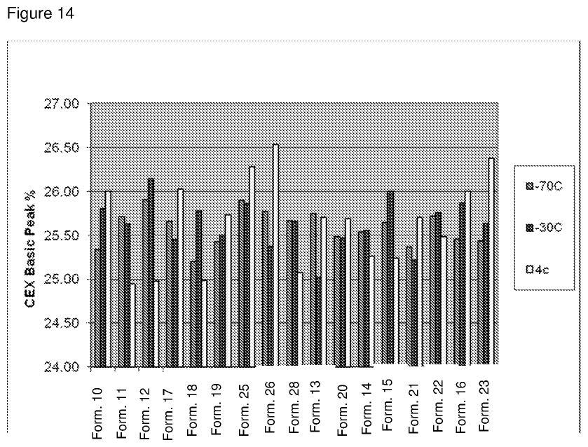

Romosozumab in A52Su was analyzed by CEX-HPLC after 3 months storage at 4° C., 25° C., and 37° C. (FIG. 11). Two-year stability data shows arginine-containing formulations, (Formulations 25 and 26) performed well, especially at 4° C., based on acidic peak data (FIG. 13). Basic peak stability data (FIG. 14) and main peak stability data (FIG. 12) are also shown for comparison.

### Capillary Electrophoresis—SDS

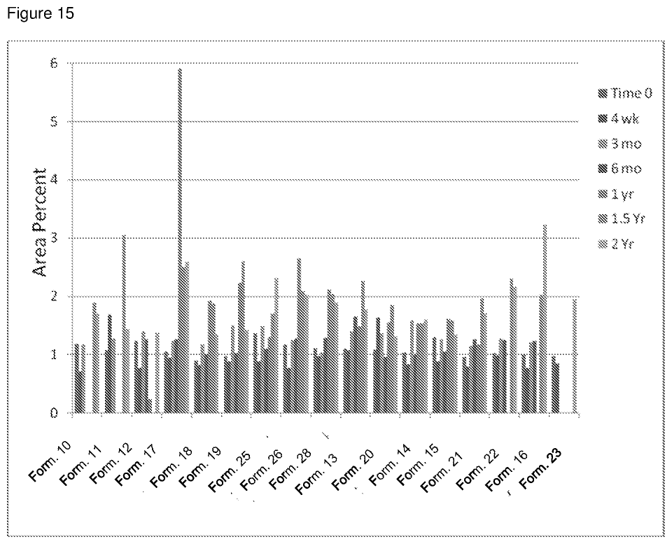

After 2 years storage at 4° C., the succinate and arginine formulations as well as the acetate at pH 4.8 formulations show the highest levels of high molecular weight species by CE-SDS as shown in FIG. 15. All samples stored for 2 years show a similar profile for % non-glycosylate heavy chain (NGHC) peak area, between 0.3-0.4% (data not shown).

In summary, the data provided in this Example demonstrates that formulations comprising arginine suppressed high molecule weigh species of romosozumab at accelerated temperatures compared to the other formulations tested.

### Example 3

### Transportation and Polysorbate 20 Concentration Study

Romosozumab in Formulation 4 was concentrated to 100 mg/mL using Millipore stirred cell (Model 8400, 400 mL capacity) with a PES membrane (10kD cutoff). Concentrated romosozumab was dialyzed into each formulation, concentrations were adjusted to 70 mg/mL with formulation buffer and polysorbate 20 was added to stated concentrations.

Samples were transported from in conditions mimicking real world transport conditions. Upon arrival, all samples were visually inspected together with the static samples prior to storage at specified temperatures, as well as freeze/thaw cycles.

TABLE 7

| Formulation | pH | Description |
|---|---|---|
| 29 | 5.2 | 10 mM Acetate, 2.5% glycerol (v/v), |
| | | 0.004% polysorbate 20 (w/v) |
| 30 | 5.2 | 10 mM Acetate, 2.5% glycerol (v/v), |
| | | 0.007% polysorbate 20 (w/v) |
| 31 | 5.2 | 10 mM Acetate, 2.5% glycerol (v/v), |
| | | 0.01% polysorbate 20 (w/v) |
| 32 | 5.2 | 10 mM Acetate, 9% sucrose (w/v), |
| | | 0.004% polysorbate 20 (w/v) |
| 33 | 5.2 | 10 mM Acetate, 9% sucrose (w/v), |
| | | 0.007% polysorbate 20 (w/v) |
| 34 | 5.2 | 10 mM Acetate, 9% sucrose (w/v), |
| | | 0.01% polysorbate 20 (w/v) |
| 35 | 5.2 | 10 mM Glutamate, 2% glycerol (v/v), |
| | | 0.004% polysorbate 20 (w/v) |
| 36 | 5.2 | 30 mM Acetate, 8.5% sucrose (w/v), |
| | | 0.004% polysorbate 20 (w/v) |
| 37 | 5.5 | 10 mM Histidine, 2.5% glycerol (v/v), |
| | | 0.004% polysorbate 20 (w/v) |
| 38 | 5.5 | 10 mM Histidine, 9% sucrose (w/v), |
| | | 0.004% polysorbate 20 (w/v) |
| 39 | 5.2 | 10 mM Acetate, 1% glycerol (v/v), |
| | | 0.1M arginine, 0.004% polysorbate 20 (w/v) |

### Size-Exclusion HPLC

Size-exclusion HPLC analysis of romosozumab shows very small differences between samples held statically in 4° C. storage compared to those which underwent real time transportation stresses before stability storage (data not shown). Different levels of polysorbate 20 performed similarly.

### Subvisible Particle Counting by Light-Obscuration (HIAC)

Formulated samples and placebos, both static and transported samples were analyzed for subvisible particles (HIAC). Notably, both the 10 μM and 25 μM count results show that higher concentrations of polysorbate 20 tend to suppress particle formation over time (Tables 8-11).

TABLE 8
10 μM Particles

| For- | T = 0 | | | | T = 1 year | | | |
|---|---|---|---|---|---|---|---|---|
| mu- | Romo | Romo | Placebo | Placebo | Romo | Romo | Placebo | Placebo |
| lation | Static | Trans | Static | Trans | Static | Trans | Static | Trans |
| 29 | 210 | 71 | 16 | 18 | 0 | 1018 | 2 | n.d. |
| 30 | 721 | 179 | 10 | 34 | 90 | 25 | 40 | n.d. |
| 31 | 565 | 135 | 4 | 21 | 15 | 22 | 37 | n.d. |
| 32 | 520 | 61 | 11 | 4 | 30 | 13 | 18 | n.d. |
| 33 | 334 | 130 | 3 | 16 | 27 | 50 | 22 | n.d. |
| 35 | 516 | 214 | 19 | 15 | 80 | 17 | 25 | n.d. |
| 36 | 550 | 160 | 6 | n.d. | 55 | 37 | 2 | n.d. |
| 37 | 475 | 476 | 5 | 5 | 68 | 32 | 25 | n.d. |
| 38 | n.d. | 419 | 4 | 35 | 50 | 122 | 43 | n.d. |
| 39 | n.d. | 355 | 6 | 15 | 122 | 25 | 30 | n.d. |

TABLE 9
10 μM Particles

| For- | T = 0 | | | | T = 2 year | | | |
|---|---|---|---|---|---|---|---|---|
| mu- | Romo | Romo | Placebo | Placebo | Romo | Romo | Placebo | Placebo |
| lation | Static | Trans | Static | Trans | Static | Trans | Static | Trans |
| 29 | 210 | 71 | 16 | 18 | 4160 | 17 | n.d. | n.d. |
| 30 | 721 | 179 | 10 | 34 | 130 | 10 | n.d. | n.d. |
| 31 | 565 | 135 | 4 | 21 | 7 | 3 | n.d. | n.d. |
| 32 | 520 | 61 | 11 | 4 | 10 | 15 | n.d. | n.d. |
| 33 | 334 | 130 | 3 | 16 | 30 | 5 | n.d. | n.d. |
| 35 | 516 | 214 | 19 | 15 | 62 | 65 | n.d. | n.d. |
| 36 | 550 | 160 | 6 | n.d. | 23 | 40 | n.d. | n.d. |
| 37 | 475 | 476 | 5 | 5 | 33 | 53 | n.d. | n.d. |
| 38 | n.d. | 419 | 4 | 35 | 28 | 45 | n.d. | n.d. |
| 39 | n.d. | 355 | 6 | 15 | 108 | 145 | n.d. | n.d. |

TABLE 10
25 μM Particles

| For- | T = 0 | | | | T = 1 year | | | |
|---|---|---|---|---|---|---|---|---|
| mu- | Romo | Romo | Placebo | Placebo | Romo | Romo | Placebo | Placebo |
| lation | Static | Trans | Static | Trans | Static | Trans | Static | Trans |
| 29 | 6 | 4 | 0 | 0 | 0 | 68 | n.d. | n.d. |
| 30 | 68 | 21 | 0 | 0 | 22 | 8 | n.d. | n.d. |
| 31 | 53 | 6 | 1 | 0 | 0 | 8 | n.d. | n.d. |
| 32 | 46 | 5 | 1 | 0 | 2 | 3 | n.d. | n.d. |
| 33 | 23 | 14 | 0 | 0 | 3 | 8 | n.d. | n.d. |
| 35 | 26 | 6 | 1 | 1 | 7 | 0 | n.d. | n.d. |
| 36 | 31 | 6 | 1 | n.d. | 2 | 3 | n.d. | n.d. |
| 37 | 23 | 35 | 0 | 0 | 10 | 2 | n.d. | n.d. |
| 38 | n.d. | 36 | 0 | 1 | 5 | 12 | n.d. | n.d. |
| 39 | n.d. | 25 | 0 | 0 | 15 | 2 | n.d. | n.d. |

TABLE 11
25 μM Particles

| For- | T = 0 | | | | T = 2 year | | | |
|---|---|---|---|---|---|---|---|---|
| mu- | Romo | Romo | Placebo | Placebo | Romo | Romo | Placebo | Placebo |
| lation | Static | Trans | Static | Trans | Static | Trans | Static | Trans |
| 29 | 6 | 4 | 0 | 0 | 218 | 0 | n.d. | n.d. |
| 30 | 68 | 21 | 0 | 0 | 25 | 0 | n.d. | n.d. |
| 31 | 53 | 6 | 1 | 0 | 0 | 0 | n.d. | n.d. |
| 32 | 46 | 5 | 1 | 0 | 2 | 2 | n.d. | n.d. |
| 33 | 23 | 14 | 0 | 0 | 3 | 0 | n.d. | n.d. |
| 35 | 26 | 6 | 1 | 1 | 8 | 7 | n.d. | n.d. |
| 36 | 31 | 6 | 1 | n.d. | 3 | 2 | n.d. | n.d. |
| 37 | 23 | 35 | 0 | 0 | 3 | 0 | n.d. | n.d. |
| 38 | n.d. | 36 | 0 | 1 | 3 | 3 | n.d. | n.d. |
| 39 | n.d. | 25 | 0 | 0 | 7 | 3 | n.d. | n.d. |

### Visual Analysis

While both size-exclusion HPLC and HIAC analysis did not indicate formulations performing better over time, visual analysis did show that certain formulations should be excluded from further study. All samples at time zero were both clear and scored 0 on visual analysis, meaning free of particles (data not shown). However, both glutamate formulations, Formulations 35 and 36 were opaque after two years storage, across temperatures, except for the −20° C. samples. Formulation 29, containing both glycerol and arginine also was opaque after two years, except for two frozen samples, one at −20° C. and one at −30° C. All samples also scored 0 (practically free of particles) at two years for visible particles (data not shown).

In summary, the data provided in this Example also demonstrates that romosozumab formulations comprising arginine were more stable under various conditions tested compared to the other formulations tested.

### Example 4

### High Concentration Syringe Study

Six syringe formulations and three vial formulations were studied in both static (not shipped) and transported (shipped) modalities. Romosozumab concentrations were 70 mg/mL and 120 mg/mL. Syringes (1 cc) were filled at 1.0 mL and vials (5 cc) at 2.0 mL. Vialed and syringe samples were shipped via a commercial domestic package carrier mimicking real world transport conditions were then stored at either 4° C. or 29° C. for up to two years.

TABLE 12
Romosozumab formulations studied.

| Formulation | Starting Buffer | Excipients and Target pH |
|---|---|---|
| 32 | 10 mM Sodum acetate | 9% sucrose, 0.004% |
| | | Polysorbate 20, pH 5.2 |
| 28 | 10 mM Sodium acetate | 8.5% sucrose, 20 mM |
| | | methionine, pH 5.2 |
| 23 | 10 mM Succinate | 9% sucrose, pH 5.2 |
| 32-1 | 10 mM Sodium acetate | 9% sucrose (w/v), 0.004% |
| | | polysorbate 20 (w/v), pH 5.2 |
| 34 | 10 mM Sodium acetate | 9% sucrose (w/v), 0.01% |
| | | polysorbate 20 (w/v) |
| 40 | 10 mM Sodium acetate | 8.5% sucrose (w/v), 20 mM |
| | | methionine, 0.004% |
| | | polysorbate 20 (w/v), pH 5.2 |
| 41 | 10 mM Sodium acetate | 8.5% sucrose (w/v), 20 mM |
| | | methionine, 0.01% |
| | | polysorbate 20 (w/v), pH 5.2 |
| 42 | 10 mM succinate | 9% sucrose (w/v), 0.004% |
| | | polysorbate 20 (w/v), pH 5.2 |
| 43 | 10 mM succinate | 9% sucrose (w/v), 0.01% |
| | | polysorbate 20 (w/v_, pH 5.2 |

### Sub-Visible Particle Analysis by Light Obscuration (HIAC)

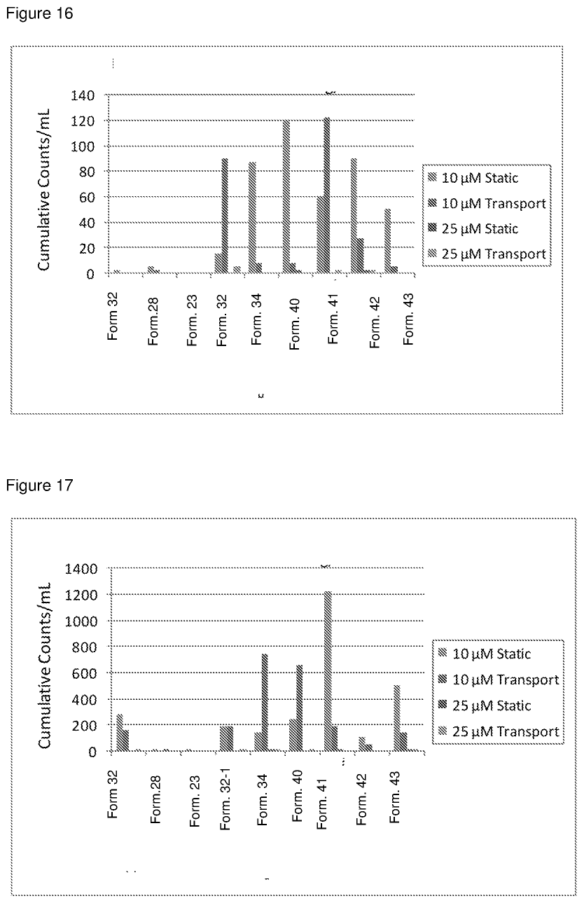

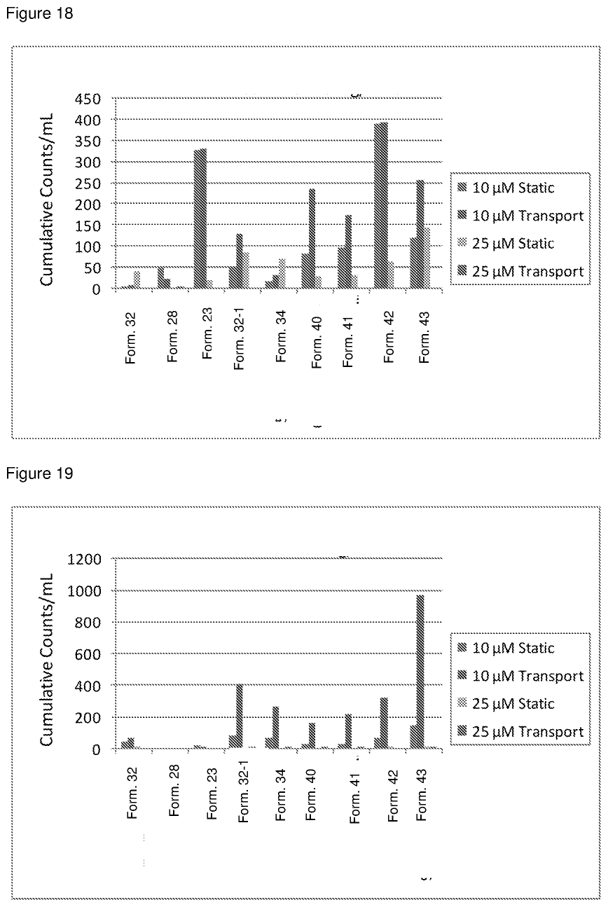

Results from the HIAC assay (light-obscuration sub-visible particle detection) showed that all protein-containing formulations, in either vial or syringe presentation were below USP guidelines for 10 μM and 25 μM particles. See FIGS. 16 through 19. For succinate formulations, 0.010% (w/v) polysorbate 20 suppressed sub-visible particle formation at 70 mg/mL but was less effective at 120 mg/mL romososumab. Vialed samples showed less sub-visible particles than syringes, irrespective of polysorbate 20 levels. In general, more sub-visible particles were detected in 120 mg/mL than 70 mg/mL.

### Visual Assay

After 2 years storage at 4° C. or 29° C., samples were assessed visually. All placebo samples in vials and syringes were clear and free of particles after 2 years. All romosozumab samples in vials and syringes were also free of particles, though a large number of samples, were either “hazy” or “cloudy” in appearance (data not shown).

Formulations that were cloudy were the succinate compositions, one in a vial, and two in syringes, and these results eliminated these formulations for further consideration. The presence of polysorbate 20 did not seem to prevent the “cloudy” result. The lower romosozumab concentration samples were “cloudy” while the 120 mg/mL samples were only “hazy.” The only samples that were more consistently “clear” after 2 years storage were the Formulation 32 vials.

### Size-Exclusion HPLC (SE-HPLC)

Size-exclusion HPLC data shows that high-molecular weight (HMW) species do increase over the 2-year period stored at 4° C. (data not shown). Romosozumab formulated at 120 mg/mL shows higher HMW generation over time as compared to the 70 mg/mL formulations at 4° C., and this is seen across all formulations studied. There are also slightly higher levels of HMW in samples that had undergone transportation stresses (data not shown). While the differences are small, Formulation 23 in both vials and syringes performed poorest, possibly due to the lack of polysorbate 20.

Data from samples stored at 29° C. for 2 years shows much higher levels of HMW species as quantified by size-exclusion HPLC as compared to 4° C. stability data (data not shown). Samples that had undergone transportation stress again showed higher levels of HMW species than the static samples. The 1.5-year time point for the 120 mg/mL HMW % results are low and out of line with the trend; this observation is true for both temperatures and static versus transported samples, so the effect may be an assay artifact.

### Cation-Exchange HPLC

Both 70 mg/mL and 120 mg/ml romosozumab protein concentrations stored statically or submitted to transportation stresses at 4° C. showed good stability using cation-exchange HPLC (data not shown).

What is claimed is:

1. A pharmaceutical composition comprising:
(a) an anti-sclerostin antibody;
(b) a buffer comprising glutamic acid, histidine or succinic acid; and
(c) a polyol,
wherein the pharmaceutical composition comprises a pH of pH4-pH7.

2. The pharmaceutical composition of claim 1, wherein the buffer is present in an amount of about 10 mM to about 50 mM.

3. The pharmaceutical composition of claim 1, wherein the polyol is present in an amount of about concentration of about 1% to about 10% w/v.

4. The pharmaceutical composition of any one of claims 1-3, wherein the polyol is sorbitol.

5. The pharmaceutical composition of claim 4, wherein sorbitol is present in an amount of about 5% to about 10% w/v.

6. The pharmaceutical composition of claim 4, wherein the sorbitol is present in an amount of about 5% w/v.

7. The pharmaceutical composition of any one of claims 1-6, further comprising glycerol.

8. The pharmaceutical composition of claim 7, wherein the glycerol is present at a concentration of about 1% to about 5% w/v.

9. The pharmaceutical composition of claim 8, wherein the glycerol is present at a concentration of about 1% w/v.

10. The pharmaceutical composition of claim 8, wherein the glycerol is present a concentration of about 2.5% w/v.

11. The pharmaceutical composition of any one of claims 1-10, further comprising sucrose.

12. The pharmaceutical composition of claim 11, wherein the sucrose is present at a concentration of about 1% to about 10% w/v.

13. The pharmaceutical composition of claim 12, wherein the sucrose is present at a concentration of about 9%.

14. The pharmaceutical composition of any one of claims 1-13, further comprising an amino acid other than histidine.

15. The pharmaceutical composition of any one of claim 14, wherein the amino acid is arginine.

16. The pharmaceutical composition of claim 15, wherein arginine is present in an amount of about 10 mM to about 250 mM.

17. The pharmaceutical composition of claim 16, wherein arginine is present in an amount of about 100 mM.

18. The pharmaceutical composition of any one of claims 1-6, 11 and 12, further comprising methionine.

19. The pharmaceutical composition of claim 18, wherein methionine is present in an amount of about 10 mM to about 100 mM.

20. The pharmaceutical composition of claim 19, wherein the methionine is present in an amount of about 20 mM.

21. The pharmaceutical composition of any one of claims 1-20, further comprising a surfactant.

22. The pharmaceutical composition of claim 21, wherein the surfactant is polysorbate 20, polysorbate 80, F16 or Triton.

23. The pharmaceutical composition of any one of claims 1-22, comprising the anti-sclerostin antibody at a concentration of at least 70 mg/mL.

24. The pharmaceutical composition of claim 23, comprising the anti-sclerostin antibody at a concentration of about 70 mg/mL to about 210 mg/mL.

25. The pharmaceutical composition of any one of claims 1-24, wherein the anti-sclerostin antibody is romosozumab.

26. The pharmaceutical composition of any one of claims 1-25, comprising 10 mM glutamic acid and 5% sorbitol at pH 4.5.

27. The pharmaceutical composition of any one of claims 1-25, comprising 10 mM glutamic acid and 5% sorbitol at pH 5.2.

28. The pharmaceutical composition of any one of claims 1-25, comprising 10 mM succinic acid and 5% sorbitol at pH 5.2.

29. The pharmaceutical composition of any one of claims 1-25, comprising 10 mM histidine and 5% sorbitol at pH 6.

30. A method of treating osteoporosis in a subject in need thereof comprising administering the pharmaceutical composition of any one of claims 1-29 to the subject.
